(19) United States
(12) Patent Application Publication
(10) Pub. No.: US 2026/0283550 A1
(43) Pub. Date: Sep. 24, 2026

(54) COUPLING PHYSIOLOGICAL MONITORS WITH WEARABLE ARTICLES

(71) Applicant: Whoop, Inc., Boston, MA (US)

(72) Inventors: Brandon Nishida-Chiou, Boston, MA (US); Mitchell Joseph Silva, Boston, MA (US); Stacey Kathleen Keetell, Maynard, MA (US); Jillian Deborah Gennaco, Manchester, NH (US)

(21) Appl. No.: 19/566,487

(22) Filed: Mar. 13, 2026

Related U.S. Application Data
(60) Provisional application No. 63/773,778, filed on Mar. 18, 2025.

(51) Int. Cl. A61B 5/00; A61B 5/0205

(52) U.S. Cl. CPC A61B 5/681; A61B 5/02055; A61B 5/6803; A61B 5/6804; A61B 2560/0214; A61B 2562/16

(57) ABSTRACT

The present teachings generally include techniques for coupling physiological monitors with garments or other wearable articles. In general, a container provides a structural and functional bridge between a wearable article and monitoring hardware, and supports integrated attachment mechanisms for removably connecting a physiological monitoring device held within the container to the wearable article, while maintaining wearer comfort, enabling accurate data collection, and facilitating easy removal and replacement. A corresponding garment may include an attachment region featuring an alignment guide for coupling with, in a removable and replaceable manner, the container holding the physiological monitor. Where the monitor and the garment communicate through RFID tags or the like, the attachment region of the garment may enforce or encourage proper relative positioning with an alignment guide.

### CROSS-REFERENCE TO RELATED APPLICATIONS

This application claims priority to U.S Provisional Patent App. No. 63/773,778 filed on Mar. 18, 2025, the entire contents of which are hereby incorporated by reference herein.

### TECHNICAL FIELD

The present disclosure generally relates to wearable physiological monitoring systems, e.g., devices, systems, and methods for coupling physiological monitors with garments and other wearable articles.

### BACKGROUND

Physiological monitoring devices have become increasingly popular for tracking various health and fitness metrics such as sleep, exercise, heart rate, and so forth. Integrating these monitors into garments in a secure yet comfortable manner can present challenges, particularly when the monitors are not integrally formed with the garment. Existing approaches for coupling physiological monitors to clothing often lack versatility in terms of placement options or use with multiple garments. There remains a need for improved techniques to securely and comfortably attach physiological monitors to garments while enabling accurate data collection and easy removal/replacement.

### SUMMARY

The present teachings generally include techniques for coupling physiological monitors with garments or other wearable articles. In general, a container provides a structural and functional bridge between a wearable article and monitoring hardware, and supports integrated attachment mechanisms for removably connecting a physiological monitoring device held within the container to the wearable article, while maintaining wearer comfort, enabling accurate data collection, and facilitating easy removal and replacement. A corresponding garment may include an attachment region featuring an alignment guide for coupling with, in a removable and replaceable manner, the container holding the physiological monitor. Where the monitor and the garment communicate through RFID tags or the like, the attachment region of the garment may enforce or encourage proper relative positioning with an alignment guide.

In an aspect, a system disclosed herein may include: a physiological monitor including one or more sensors on a sensing surface thereof and an RFID reader; a garment with an RFID tag, a first hook and loop fastener over the RFID tag, and an alignment guide around the first hook and loop fastener; and a container for removably and replaceably attaching the physiological monitor, the container including a rigid skeleton and a deformable shell overmolded onto the rigid skeleton. The deformable shell may include: an interior space shaped and sized to receive the physiological monitor, an opening to the interior space on a first side of the container, the opening positioned to expose the one or more sensors through the opening when the physiological monitor is placed for use in the interior space, and a second hook and loop fastener on a second side of the container, the second side of the container shaped and sized to fit within the alignment guide of the garment in a predetermined position and orientation when the first hook and loop fastener is coupled to the second hook and loop fastener, where the predetermined position and orientation between the alignment guide and the second side of the container orients the RFID reader of the physiological monitor to retrieve data from the RFID tag of the garment.

The one or more sensors of the physiological monitor may include one or more optical sensors. The one or more sensors of the physiological monitor may include one or more electrical sensors. The one or more sensors of the physiological monitor may include one or more temperature sensors. The alignment guide may include a raised border forming an opening providing a mechanical key to align the container. The RFID tag may encode identifying information for the garment including at least one of: a location of an attachment region on the garment, a location of the attachment region relative to a body of a wearer when placed for use, a type of the garment, a size of the garment, and one or more preferred sensing operations for the garment or attachment region location. The container may further include a tab extending from a sidewall of the deformable shell, the tab providing an alignment key for enforcing the predetermined position and orientation of the container within the alignment guide. The rigid skeleton may extend beyond the interior space of the deformable shell in at least one dimension, thereby providing a rigid structure to the container extending beyond the physiological monitor in the at least one dimension when the physiological monitor is placed for use in the interior space. The at least one dimension may be the longest of three orthogonal dimensions passing through a point in the interior space. The rigid skeleton may be formed of a polycarbonate. The deformable shell may be formed of a silicone. The deformable shell may have a thickness between the interior space and the second side of the container not greater than a sensing distance for the RFID tag to be sensed by the RFID reader when the physiological monitor is placed for use in the interior space. The second side of the container may have a curvature with a center of curvature in a location toward the first side of the container from the second side of the container. The garment may include at least one of a shirt, pants, a compression sleeve, and an undergarment. The container may include one or more extensions that extend from a body of the container over at least a portion of the opening, where the one or more extensions are deformable to allow the physiological monitor to pass through the opening and to conform around the physiological monitor when disposed within the container. The physiological monitor may protrude a predetermined distance beyond the first side of the container when disposed within the container. The deformable shell may be formed at least in part using one or more of: thermoplastic elastomer, thermoplastic vulcanizates, ethylene propylene diene monomer, or rubber. The rigid skeleton may include one or more of: acrylonitrile butadiene styrene (ABS), acrylic, polybutylene terephthalate (PBT), or styrene ethylene butadiene styrene (SEBS). The deformable shell may have a predetermined tackiness that resists lateral movement of the container relative to skin of a wearer. The garment may include a substantially elastic material structurally configured to apply a normal force directing the container and the physiological monitor into skin of a wearer, where the normal force results from hoop stress created by the garment urging the container toward the skin. The container and the physiological monitor may be collectively structurally configured to create an indent into tissue of a wearer while wearing the garment. The container may include an insert configured to snap-fit, adhere, or mechanically lock into the rigid skeleton, where the insert includes the second hook and loop fastener on an exterior surface thereof. The system may further include a charging component configured to wirelessly charge the physiological monitor when the physiological monitor is placed for use in the interior space. The alignment guide may include a raised border defining a physical barrier that abuts sidewalls of the container to inhibit lateral shear movement and rotation of the container relative to the garment. Connection between the container and the physiological monitor may include one or more of: a snap fit, a friction fit, mechanical keying between features thereof, or magnetic attraction. The container may elastically deform to receive and retain the physiological monitor by stretching around the physiological monitor as the physiological monitor is inserted therein.

In an aspect, a method disclosed herein may include inserting a physiological monitor having one or more sensors on a sensing surface thereof into a container, the container including: a first side and a second side, the first side defining an opening to an interior space shaped and sized to receive the physiological monitor, the second side including a fastener; a rigid skeleton disposed at least along the second side of the container to form a backbone structure thereof; and a deformable shell overmolded onto the skeleton and defining at least a portion of a perimeter of the opening to the interior space, where the sensing surface is exposed through the opening when the physiological monitor is disposed within the container. The method may further include: coupling the second side of the container to an attachment region of a wearable article, the attachment region including a complementary fastener to engage with the fastener of the container, the attachment region further including an alignment guide to provide a predetermined position and orientation of the container when coupled to the attachment region; wearing the wearable article such that the sensing surface is disposed adjacent to skin of a wearer; and performing a sensing operation using the one or more sensors of the physiological monitor.

Coupling the second side of the container to the attachment region of the wearable article may include aligning the container with the alignment guide. The alignment guide may include a raised border having one or more features providing a mechanical key, where aligning the container includes engaging a corresponding feature of the container with the mechanical key. The attachment region of the wearable article may include a data tag, where the method further includes reading data from the data tag using a data reader of the physiological monitor. The data may include at least one of location of the attachment region on the wearable article, location of the attachment region relative to the wearer, information regarding the wearer, and information regarding the wearable article. The method may further include adapting operation of the physiological monitor based on the data. Adapting operation may include selecting one or more of a filter, a processing model, or a physiological signal detection algorithm based on the data. The method may further include generating a normal force pressing the container toward the skin of the wearer via hoop stress created by the wearable article. The method may further include creating an indent into tissue of the wearer with the container and the physiological monitor for optical or electrical coupling between the one or more sensors and target tissue. Inserting the physiological monitor into the container may include elastically deforming one or more extensions of the container to allow the physiological monitor to pass through the opening, where the one or more extensions elastically return to conform around the physiological monitor to retain the physiological monitor within the container. The method may further include removing the physiological monitor from the container, and replacing the physiological monitor to the container. The sensing operation may include acquiring heart rate data using photoplethysmography. The method may further include transmitting data acquired during the sensing operation to a remote processing resource for analysis. The deformable shell may contact the skin of the wearer and resists lateral movement of the container relative to the skin due to a tackiness of the deformable shell.

In an aspect, a wearable article disclosed herein may include: a sheet of material forming at least a portion of the wearable article; a radio frequency identification (RFID) tag disposed on the sheet of material, the RFID tag encoding identifying information for the wearable article; a hook and loop fastener disposed over the RFID tag; and an alignment guide including a raised border around the hook and loop fastener, the raised border including one or more features providing a mechanical key to enforce a predetermined position and orientation of a device with a corresponding shape placed thereon.

The wearable article may include at least one of a shirt, pants, a compression sleeve, and an undergarment. The raised border of the alignment guide may form an open C with an opening providing the mechanical key to align the device. The identifying information may include at least one of: a location of the wearable article on a body of a wearer when placed for use, a type of the wearable article, a size of the wearable article, and one or more preferred sensing operations for the wearable article. The raised border may define a physical barrier structurally configured to abut sidewalls of a container to inhibit lateral shear movement and rotation of the container relative to the wearable article. The sheet of material may include a substantially elastic material structurally configured to apply a normal force directing a container coupled thereto toward skin of a wearer. The sheet of material may include at least one of polyester, spandex, nylon, wool, bamboo fiber, or polypropylene. The wearable article may include a plurality of attachment regions, each attachment region including a respective RFID tag, fastener, and alignment guide. Each RFID tag of the plurality of attachment regions may encode a unique location identifier corresponding to a respective body location when the wearable article is worn. The RFID tag may be disposed between the sheet of material and the hook and loop fastener. The raised border may be formed of a material that is more rigid than the sheet of material. The alignment guide may be disposed on an interior surface of the wearable article configured to face skin of a wearer when the wearable article is worn. The wearable article may include a fabric patch attachable to a garment, the fabric patch including the sheet of material, the RFID tag, the hook and loop fastener, and the alignment guide.

In an aspect, a container disclosed herein for a physiological monitor may include: a skeleton of a rigid material; a deformable shell overmolded onto the skeleton, the deformable shell having a first side and a second side where the deformable shell defines an interior space shaped and sized to retain the physiological monitor, the interior space includes an opening on the first side of the deformable shell for removal of the physiological monitor from, and replacement of the physiological monitor to, the interior space, the second side of the deformable shell has a curvature with a center of curvature in a location toward the first side of the deformable shell from the second side of the deformable shell, and the opening is positioned to expose one or more sensors of the physiological monitor through the opening for physiological sensing when the physiological monitor is placed for use in the interior space of the container and the container is placed for use on a user; and a hook and loop fastener disposed on the second side of the deformable shell.

The container may further include a tab extending from a sidewall of the deformable shell, the tab providing an alignment key for enforcing a predetermined position and orientation of the container in a guide. The skeleton may extend beyond the interior space of the deformable shell in at least one dimension, thereby providing a rigid structure to the container extending beyond the physiological monitor in the at least one dimension when the physiological monitor is placed for use in the interior space of the container. The at least one dimension may be the longest of three orthogonal dimensions passing through a point in the interior space. The skeleton may be formed of a polycarbonate. The deformable shell may be formed of a silicone. The deformable shell may have a thickness between the interior space and the second side of the deformable shell not greater than a sensing distance for an RFID tag to be sensed by the physiological monitor when placed for use in the interior space of the container. The deformable shell may include one or more extensions that extend from a body of the container over at least a portion of the opening, where the one or more extensions are deformable to allow the physiological monitor to pass through the opening and to conform around the physiological monitor when disposed within the container. The skeleton may be longer than a length of the opening of the container. The skeleton may be wider than a width of the opening of the container. The deformable shell may have a predetermined tackiness that resists lateral movement of the container relative to skin of a wearer when the container is placed for use on the wearer. The container may include one or more engagement features disposed in the interior space, the one or more engagement features configured to align, protect, or secure the physiological monitor within the container. The one or more engagement features may include at least one of a projection or an indentation that cooperates with a corresponding structural element on the physiological monitor. The skeleton may include one or more extensions that serve as anchor points for securing the deformable shell onto the skeleton. The skeleton and the deformable shell may be formed of materials that are substantially radio-frequency transparent at operating frequencies of an RFID tag and reader. The container may further include an insert configured to snap-fit, adhere, or mechanically lock onto the skeleton, wherein the insert includes the hook and loop fastener on an exterior surface thereof. The container may further include a charging component configured to wirelessly charge the physiological monitor when the physiological monitor is placed for use in the interior space. The physiological monitor may protrude a predetermined distance beyond the first side of the deformable shell when disposed within the interior space. The skeleton may be formed of a material that is more rigid than the deformable shell. The skeleton may include one or more of acrylonitrile butadiene styrene (ABS), acrylic, polybutylene terephthalate (PBT), or styrene ethylene butadiene styrene (SEBS). The deformable shell may be formed at least in part using one or more of thermoplastic elastomer, thermoplastic vulcanizates, ethylene propylene diene monomer, or rubber. The curvature of the second side of the deformable shell may have a radius of curvature selected to create a predetermined hoop stress when the container is coupled to a wearable article worn by a user. Connection between the container and the physiological monitor may include one or more of a snap fit, a friction fit, or mechanical keying between features thereof. A depth of the interior space may be shorter than a height of the physiological monitor such that the physiological monitor projects at least partially from the opening when fully seated within the container.

In an aspect, a method of manufacturing a container for physiological monitors may include: forming a skeleton of a substantially rigid material; overmolding at least a portion of the skeleton with a substantially deformable material to provide a deformable shell, the deformable shell having sidewalls extending from the skeleton that at least partially define an interior space shaped and sized to retain a physiological monitor; forming an opening on a first side of the container for insertion and removal of the physiological monitor; and attaching a fastener to a second side of the container opposing the first side.

Forming the skeleton may include injection molding the substantially rigid material. The method may further include forming one or more extensions that extend over at least a portion of the opening. The substantially rigid material may include polycarbonate, and wherein the substantially deformable material includes silicone. The method may further include forming a tab extending from a sidewall of the deformable shell. The skeleton may include a length that extends beyond the interior space in at least one dimension. The fastener may include a hook and loop fastener. The method may further include attaching an insert to the skeleton, wherein the insert includes the fastener on an exterior surface thereof.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 shows a system for coupling a physiological monitor with a wearable article.

FIG. 7A shows a top view of an attachment region of a wearable article.

FIG. 7B shows a side view of an attachment region of a wearable article.

FIG. 7C shows a front view of an attachment region of a wearable article.

FIG. 7D shows a rear view of an attachment region of a wearable article.

FIG. 8 shows an isometric view of a container for a physiological monitor.

FIG. 9 shows a side view of a container for a physiological monitor.

FIG. 10 shows a bottom view of a container for a physiological monitor.

FIG. 11 shows perspective bottom views of a container for a physiological monitor.

FIG. 12 shows a system for physiological monitoring.

FIG. 13 shows portions of a container for a physiological monitor.

FIG. 14 shows portions of a container for a physiological monitor.

FIG. 15 shows a container with a semi-translucent shell.

FIG. 16 is a flow chart of a method for coupling a physiological monitor with a wearable article.

FIG. 17 is a flow chart of a method of manufacturing a container for physiological monitors.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods such as an hour, a day or more (including acquisition throughout the day and night), etc. More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example increasing the sampling rate when signal variability increases, or during periods of relatively higher motion, or based on user input.

At the same time, continuous monitoring is not intended to exclude ordinary data acquisition interruptions such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data such as optical intensity signals, or processed data therefrom such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component or module of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as, DRAM, SRAM, EDO RAM), and so forth. Although not depicted, any of the devices or components described herein may include a computer-readable medium or other memory for storing program instructions, data, and the like.

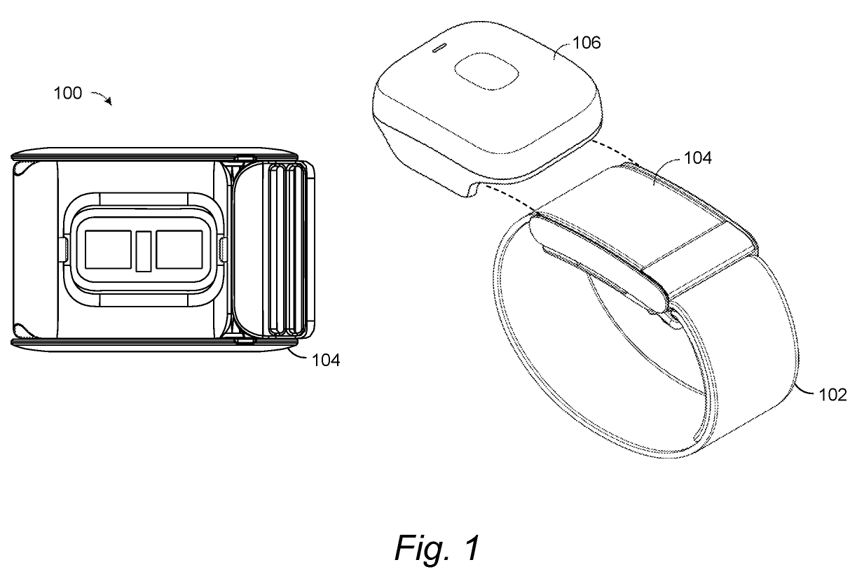

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer’s body for the acquisition of physiological data as described herein. For example, the strap 102 may include a slim elastic band formed of any suitable elastic material such as a rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user’s body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. United States Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104 such as data collection, processing, display, and communications with external resources. For example, the system 100 may include hardware for a heart rate monitor using, e.g., photoplethysmography, electrocardiography, or any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light emitting diodes (LEDs)) and optical detectors in the wearable monitor 104. The LEDs may be positioned to direct illumination toward the user’s skin, and optical detectors such as photodiodes may be used to capture illumination intensity measurements indicative of illumination from the LEDs that is reflected and/or transmitted by or through the wearer’s skin, or depending on the configuration, through capillaries or arteries.

The system 100 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure (via physical pressure measurements or other means), sound, electrocardiograms, and the like, as well environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting addition functions of the wearable monitor 104. For example, the system 100 may include communications systems to support, e.g., near field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a GeoPositioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

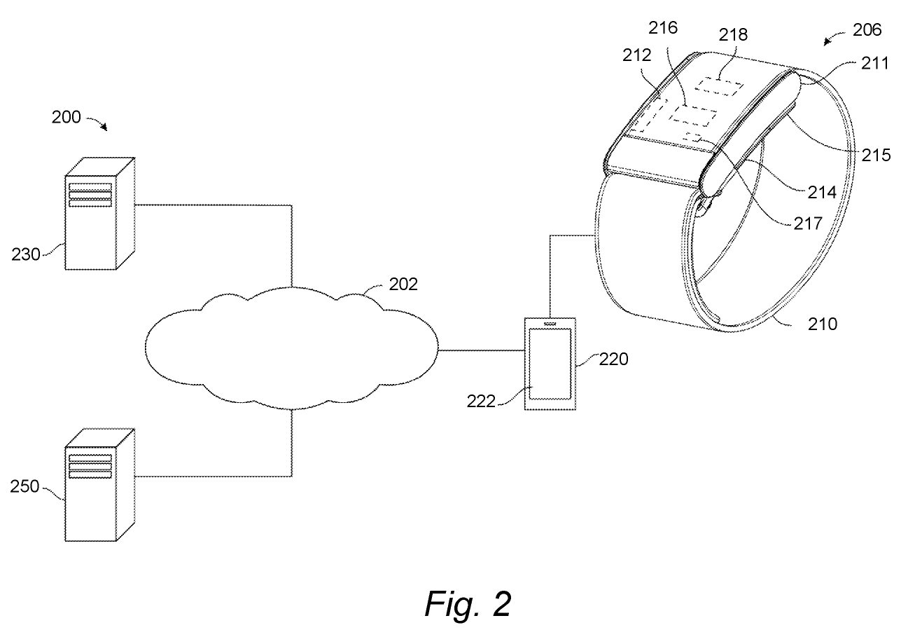

FIG. 2 illustrates a physiological monitoring system. More specifically, FIG. 2 illustrates a system 200 for physiological monitoring that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communicating with local resources. By way of non-limiting examples, short range communications may include Wi-Fi communications, Bluetooth communications, infrared communications, near field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicated detected motion, and/or beacons or the like to self-identify type, weight, and so forth, in order to facilitate automated detection and tracking of exercise activity with other connected devices.

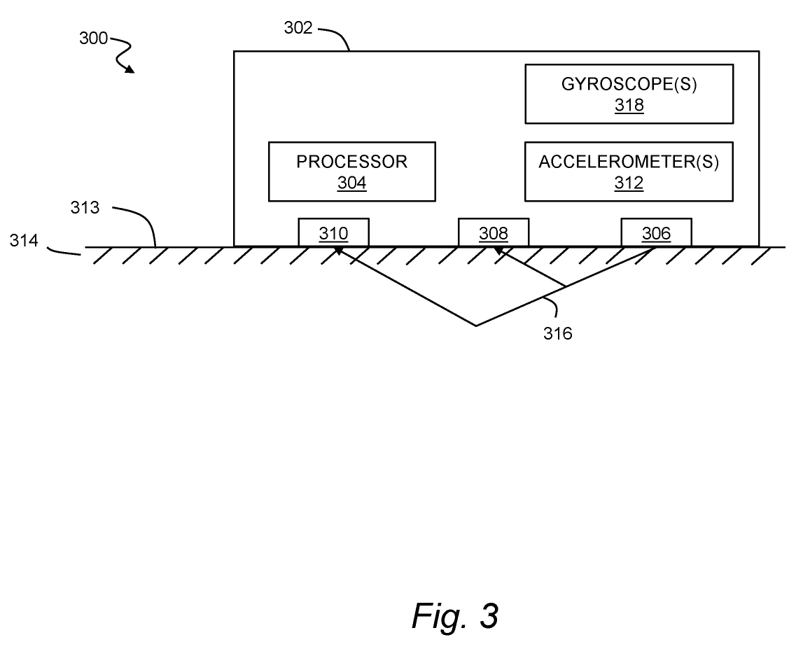

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like such as audio earbuds, earrings or similar, glasses and/or other eyewear, a ring, a headband, and so forth.

The processor 304 may be any microprocessor, microcontroller, application specific integrated circuit, or other processing circuitry or combination of the foregoing suitable for controlling operation of the physiological monitor and acquiring physiological data.

The light source 306 may include one or more light emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light emitting diodes that emit light in the green, red, infrared, near infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1–4 millimeters from the light source 306 and the second sensor 310 may be positioned 2–8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors 308 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

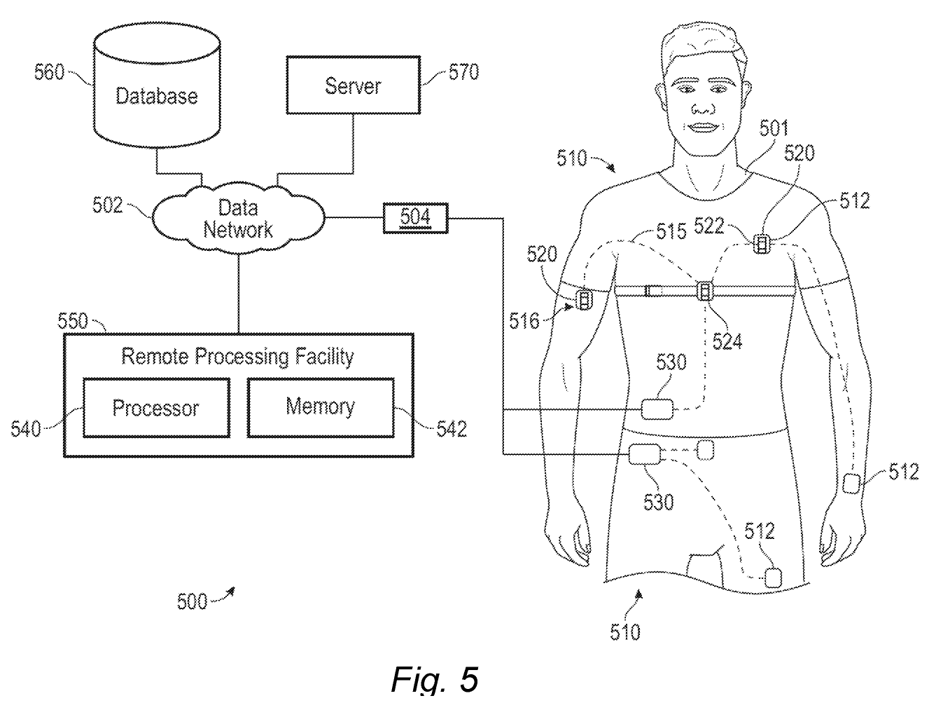

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

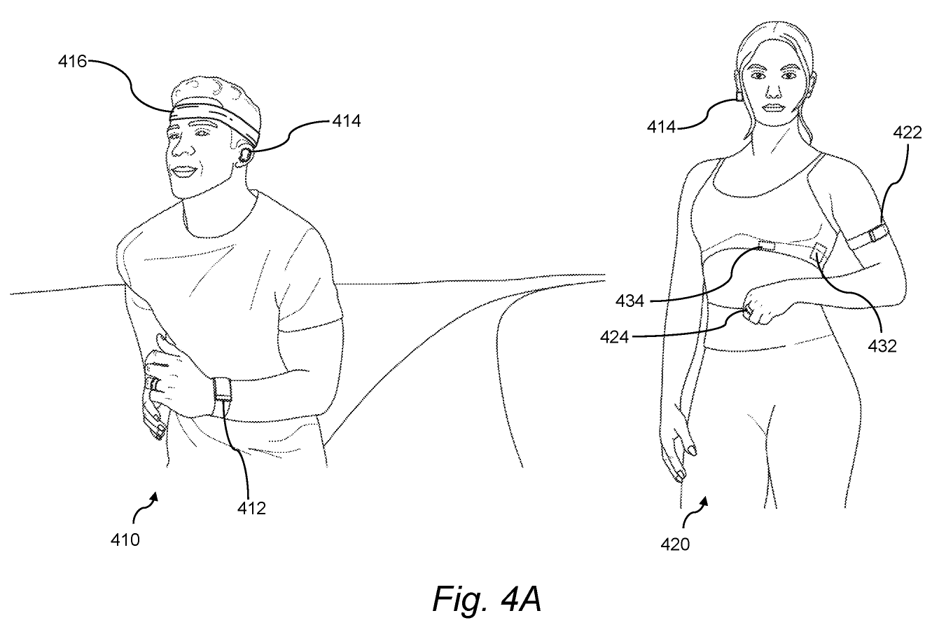

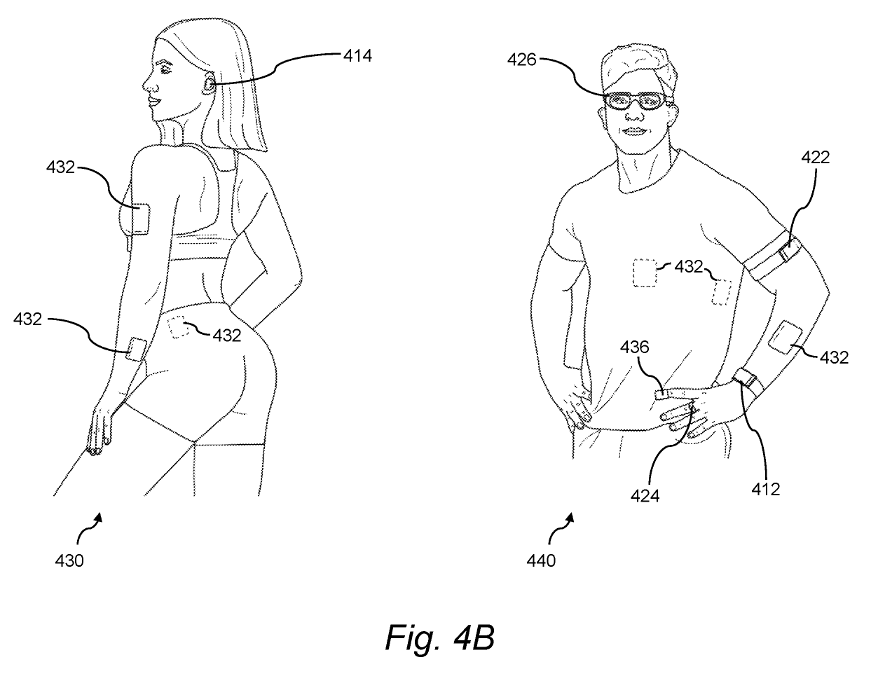

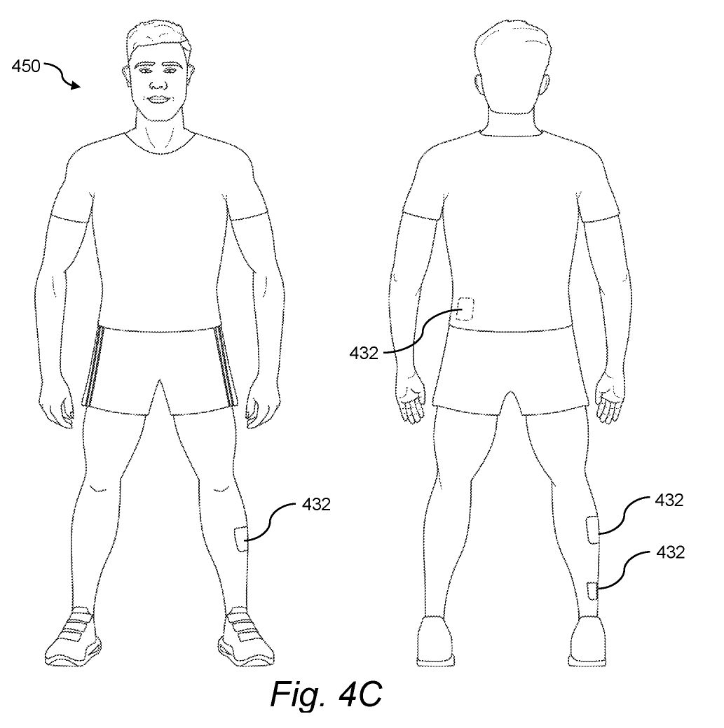

FIGS. 4A–4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1–3 above.

FIG. 4A shows a first user 410 and a second user 420. The first user 410 may be wearing one or more physiological monitors such as a wrist-worn device 412 (such as any described herein), an ear-worn device 414 (including on-ear devices retained with a clamp, clip, or other mechanism, and/or in-ear devices such as earbuds or the like that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large scale motion such as geographical movement and elevation changes that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors, and may be more suited to, e.g., wrist bands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to a pulse oximetry or SpO₂) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer’s body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smart phone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a finger-tip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The finger-tip monitor 436 may include a clamp, clip, or the like, and may be the same or similar to any of the physiological monitors described herein. In some aspects, the finger-tip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smart phone or other computing device, to perform cooperative monitoring. For example, various monitoring techniques, such as electrocardiography or blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as wi-fi or cellular signals, and converting them into usable electrical power), ambient light harvesting, and so forth. Other techniques may also or instead be used to provide external power, such as beam steering or resonant techniques for short range or medium range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user’s body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system to be embedded in a sleeve cuff for interactions with a payment terminal. Smart garments may also free up body surfaces for other devices. For example, if sensors in a wrist-worn device that provide heart rate monitoring and step counting can be instead embedded in a user’s undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smart phone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interactions, for example by storing data locally and transmitting the data over available local area network resources when a local access point such as a wireless access point or a relay device (such as a laptop, tablet, or smart phone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within the one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user’s skin, and one or more light detectors for received light reflected from the user’s skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities while heart rate data may preferentially be gathered near major arteries. In another aspect, some measurements such as temperature may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly where packet loss, latency, and the like may present challenges to real time processing.

The communications interface 524 may be any as described herein, for example including any of the features of the network interface 504 described above.

The controller 530 may be configured, e.g., by computer executable code or the like, to determine a location of the module 520. This may be based on contextual measurements such as accelerometer data from the module 520, which may be analyzed by a machine learning model or the like to infer a body position. In another aspect, this may be based on other signals from the module 520. For example, signals from sensors such as photodiodes, temperature sensors, resistors, capacitors, and the like may be used alone or in combination to infer a body position. In another aspect, the location may be determined based on a proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500 such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed such as machine learning models, lookup tables, analytical models, or the like, which may be applied to accelerometer data to detect an activity type. Other motion data such as gyroscope data may also or instead be used, and activity recognition processes may also be augmented by other potentially relevant data such as data from a barometer, magnetometer, GPS system, and so forth. This may generally discriminate, e.g., between being asleep, at rest, or in motion, or this may discriminate more finely among different types of athletic activity such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes, and may, for example, be used to improve the design or use of modules 520 and garments 510, either for a population, for a user type, or for a particular user.

As stated above, the system 500 may further include a processor 540 and a memory 542. In general, the memory 542 may bear computer executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like as shown in the figure. Thus, in an aspect, one or more of the processor 540 and the memory 542 is included on at least one of the plurality of modules 520. In this manner, processing may be performed on a central module, or on each module 520 independently. In another aspect, one or more of the processor 540 and the memory 542 is remote relative to each of the plurality of modules 520. For example, processing may be performed on a connected peripheral device such as smart phone, laptop, local computer, or cloud resource.

The processor 540 may be configured to assess the quality of the data received from a physiological sensor 522 of the module 520, otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources, may advantageously be used in combination to improve physiological monitoring and achieve modes of monitoring not previously available.

In general, the devices, systems, and methods disclosed herein relate to coupling physiological monitors with garments and other wearable articles. It will be understood that the terms “garment” and “wearable article” (or simply “article”) may be used interchangeably herein, as the present teachings may be applied to either or both without further adaptation. Thus, where one term is used, the other will be interpreted as being included as well, unless expressly stated to the contrary. In general, a garment may include an item of clothing, such as any described above—e.g., tops, bottoms, undergarments, overgarments, swimwear, and the like. In general, wearable apparel may include any wearable item or accessory—e.g., bands (arm bands, chest bands, leg bands, and so forth), a compression sleeve, a brace, padding, sports or training equipment, and so forth.

Several challenges may exist when attempting to incorporate physiological monitors into garments. There may be comfort issues—monitors may be rigid and uncomfortable when integrated into clothing; less comfortable tight-fitting garments may be needed or monitors may be placed in tight-fitting regions of a garment, such as a waistband; the skin-sensor interface can cause discomfort; and the like. For example, physiological monitors can be incorporated into elastic band portions (e.g., a waistband), but this can create discomfort for some wearers. Moreover, this can limit the location for placement of physiological monitors, and these limited locations may not always be ideal for sensing certain physiological signals—by way of example, the waistband area may yield “noisy” sensed data relative to other areas such as the glute region as it relates to a signal-to-noise ratio (SNR) or similar. There may also or instead be movement and/or deformation issues—monitors may be subjected to mechanical deformations of a garment during body movement, compromising their sensing capability; the skin-sensor interface can experience shifting, where sensors lose proper sensing contact with a user; the skin-sensor interface can cause wearer discomfort; and so forth. Further, there may be limitations on interchangeability, removability, adaptability, and the like.

The present teachings may address one or more of the aforementioned concerns. More specifically, the present teachings may relate to wearable articles with integrated attachment mechanisms for securely and removably connecting monitoring devices inserted within containers. In aspects, the attachment region on a wearable article may include an alignment guide providing a mechanical key to enforce a position and orientation of the container, and/or to provide a visual guide for a user, where the container includes a corresponding shape structurally configured to cooperate with the alignment guide. Such alignment may promote advantageous sensing, provide comfort for a wearer, provide an easy guide for proper attachment by a user, and/or enable communication (or other data transmission) between components of the attachment region and the physiological monitor. For example, certain aspects of the present teachings include a radio frequency identification (RFID) tag present within the attachment region and an RFID reader present on the physiological monitor, where it will be understood that other such communication components may also or instead be utilized. In this manner, the present teachings may include determining the location of a physiological monitor on a garment and/or adapting operation based on detected location, where such detection is provided by the predetermined engagement and alignment via the cooperation of the attachment region and the container. By way of example, a wearable article may include a sheet of material, such as a fabric or textile, that forms at least a portion of the article, where an RFID tag or similar may be disposed on the sheet of material, and may encode identifying information for the wearable article, such as a unique identifier, a location on the body, or other relevant data. A fastener such as a hook and loop fastener may be disposed over the RFID tag, and may be used to secure the container to the wearable article. An alignment guide may be disposed around the hook and loop fastener, and may include a raised border or other feature that provides a mechanical key to enforce a predetermined position and orientation of the container.

In this manner, the present teachings may include a system featuring a physiological monitor that is configured to acquire physiological data from a user (such as any as described herein), a garment, and a container for holding the monitor and for placement on the garment. The garment may include an RFID tag or similar that encodes identifying information, such as a unique identifier or location on the body, and may be used to communicate with the physiological monitor. The container may include a rigid skeleton that provides structural support and stability, and a deformable shell that is overmolded onto the skeleton. The container may be configured to receive the physiological monitor, and may include a hook and loop fastener that secures the container to the garment. The system may be configured to orient the RFID reader of the physiological monitor to retrieve data from the RFID tag of the garment, and may use this data to adapt operation of the physiological monitor or provide feedback to the user.

Thus, the present teachings may advantageously provide for physiological monitors to be properly and reliably secured to wearable articles in a manner that maintains good contact between sensors and target surfaces while maintaining wearer comfort, enabling accurate data collection, and facilitating easy removal and/or replacement. As a significant advantage, the techniques described herein can expand the range of possible sensing locations by providing a retaining structure optimized for sensor contact and positional stability during physical movement by the user.

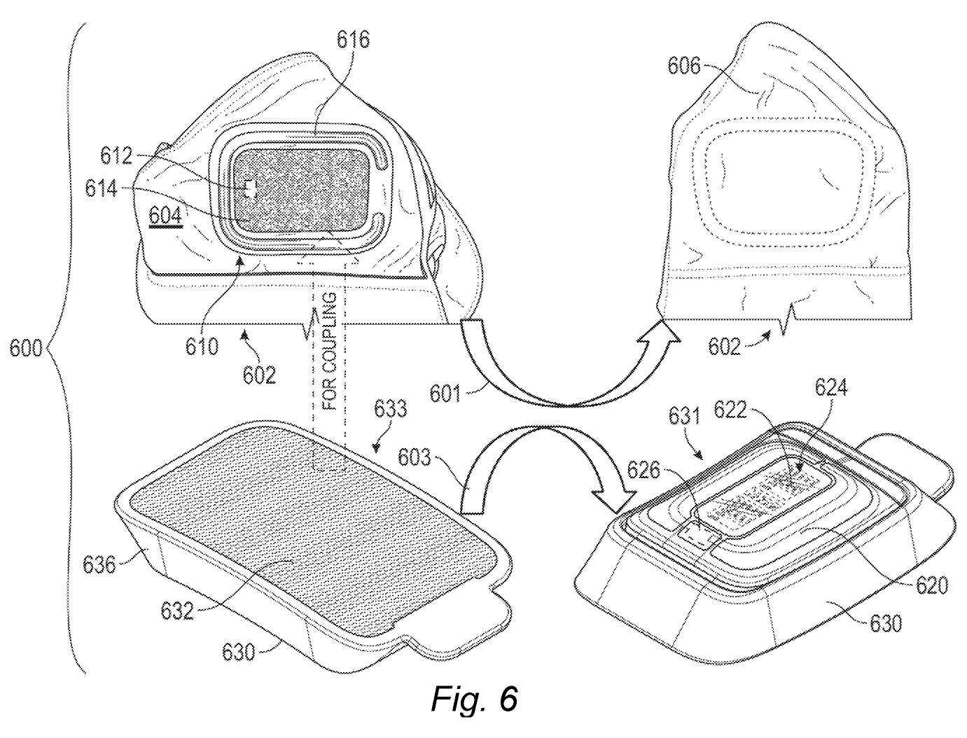

FIG. 6 shows a system for coupling a physiological monitor with a wearable article. It will be understood that aspects of the system 600 of FIG. 6 may be utilized with one or more of the devices and systems shown and described with respect to FIGS. 1–5 above, and vice-versa. By way of example, the modules 520 of FIG. 5 may correspond to the monitor 620 of FIG. 6, and the designated areas 512 of FIG. 5 may correspond to the attachment region 610 of FIG. 6. By way of further example, the physiological monitor 206 of FIG. 2 may correspond to the monitor 620 of FIG. 6—e.g., where a monitor 620 of FIG. 6 is couplable to a strap such as that shown in FIG. 2. The system 600 may generally include a garment 602, an attachment region 610, a monitor 620, and a container 630.

The garment 602 may include any as described herein, including any wearable article or the like. The garment 602 may include an interior surface 604 and an exterior surface 606 disposed opposite the interior surface 604. The interior surface 604 may include at least a portion that is positioned against, or closely adjacent to, a wearer’s skin when the garment 602 is donned by or otherwise placed for use on a user. For ease in understanding, the figure includes a first arrow 601 indicating the reverse side of the image, i.e., pointing from the interior surface 604 of the garment 602 to the exterior surface 606 of the garment 602. The garment 602, and more specifically the interior surface 604 of the garment 602, may include one or more attachment regions 610 disposed thereon. Such attachment regions 610 may be integral with the garment 602, and/or may be removable and replaceable with respect to the garment 602—e.g., where an attachment region 610 can be removed, moved from a first location on the garment 602 to a second location, and so forth.

The garment 602 may further include an RFID tag 612 or the like, disposed on or within the attachment region 610 of the garment 602. The RFID tag 612 may generally be configured to exchange data with an RFID reader 626, e.g., included on or within the monitor 620 and/or the container 630. This data transmission may be one-way (e.g., from the RFID tag 612 to the RFID reader 626) or bi-directional. The RFID tag 612 and/or RFID reader 626 may be disposed in specific, predetermined locations on their respective components. Although this disclosure emphasizes the use of RFID technology for communication between the garment 602 and the monitor 620 (and/or the container 630), it will be understood that alternative communication, data storage, identification, and/or data transmission technologies and protocols may also or instead be utilized. By way of example, the RFID components described herein may be replaced by, or supplemented with, one or more of the following: Bluetooth low energy devices, code readers (e.g., QR codes), near field communication (NFC) devices, computer vision, infrared devices, low-power wireless communication devices, and the like. Thus, where the terms “tag” and “reader” are used, it will be understood to include other similar data transmission and communication technologies unless expressly stated to the contrary. In another aspect, data may be mechanically, magnetically, optically, or otherwise encoded in the garment 602 in a manner that can be detected and decoded by a corresponding sensor or reader in a device adjacent thereto. It will also be understood that either or both of a “tag” and “reader” may have “write” capabilities in addition to or instead of “read” capabilities. It will be further understood that the location of these components—the RFID tag 612 and the RFID reader 626—are provided by way of representation and example, and that other locations are also or instead possible. For example, the RFID reader 626 may be disposed in the monitor 620 toward a surface that opposes the sensing surface 624, which may be advantageous as this surface would be disposed closer to the RFID tag 612 when the container 630 is coupled to the attachment region 610. Also or instead, the RFID tag 612 may be disposed substantially in the center of the attachment region 610, e.g., under the first fastener 614. In another aspect, the RFID tag 612 may be positioned on the monitor 620 and the corresponding RFID reader 626 may be positioned on the garment 602, e.g., where the garment 602 is a smart garment containing power, communications, and or processing infrastructure for physiological monitoring. In certain aspects, the positions of these components may be arranged such that they are substantially aligned or otherwise disposed relatively near one another in a location suitable for data transmission therebetween, when the container 630 is coupled to the attachment region 610.

The garment 602 may include a first fastener 614, disposed on or within the attachment region 610 of the garment 602 as described herein. The first fastener 614 may be structurally configured to engage with a second fastener 632 disposed on the container 630 as described herein. In certain aspects, these fasteners may include hook and loop fasteners. For example, the first fastener 614 may include the loop portion of a hook and loop fastener, while the second fastener 632 may include the hook portion of a hook and loop fastener. This arrangement may be particularly advantageous because the first fastener 614 may contact a wearer’s skin when the garment 602 is being worn and is uncoupled from the container 630, and the loop portion may provide a soft and comfortable feel against the skin relative to a rougher hook portion of a hook and loop fastener. Other fasteners in addition to or instead of hook and loop fasteners may be used including one or more of the following: snaps, magnets, buttons, other mechanical keying components, clamps, clips, dowels, friction fits, snap fits, adhesive, latches, pins, screws, sliders, and the like. The first fastener 614 may be disposed over the RFID tag 612 on the attachment region 610 of the garment 602.

The garment 602 may include an alignment guide 616, disposed on or around the attachment region 610 of the garment 602 as described herein. The alignment guide 616 may be structurally configured to align the container 630 in a predetermined position and/or orientation relative to the garment 602, and more specifically the attachment region 610 of the garment 602. The alignment guide 616 may be disposed at least partially around the first fastener 614.

The monitor 620 may be any as described herein, e.g., a wearable physiological monitor that is configured for physiological monitoring using one or more sensors 622. The sensors 622 may be any as described herein—e.g., utilizing photoplethysmography or similar, where a pulse or heart rate may be acquired optically based on a light source (e.g., one or more LEDs) and one or more optical detectors in the monitor 620 or using any other electrical, optical, mechanical, magnetic, chemical, or other sensing modalities to track physiological information of interest. The physiological sensing and/or monitoring conducted by the monitor 620 may more generally include any as described herein, e.g., with reference to the description of the system 100 of FIG. 1 above. By way of example, the one or more sensors 622 may include at least one of the following: one or more optical sensors, one or more electrical sensors, one or more temperature sensors, and the like.

The monitor 620 may further include a sensing surface 624, upon which or through which the one or more sensors 622 detect physiological signals. The sensing surface 624 may be structurally configured to touch or rest against skin of a user to perform sensing using the one or more sensors 622, and/or, depending on the sensing modality, to be disposed sufficiently close to the skin of a user to support monitoring (e.g., within 1mm or less).

The container 630 may be structurally configured for removably and replaceably receiving the monitor 620. For example, the container 630 may be structurally configured to hold the monitor 620 at least partially therein, and may include an interior space shaped and sized to receive at least a portion of the monitor 620. The connection between the container 630 and the monitor 620 may include one or more of the following: a snap fit, a friction fit, mechanical keying between features thereof, magnetic attraction, and the like. In another aspect, the container 630 may elastically deform to receive and retain the monitor 620 by stretching the container 630 around the monitor 620 as the monitor 620 is inserted therein. The connection may also or instead include any of the fastening means described herein, e.g., with respect to the first and second fasteners 614, 632 or otherwise. The connection between the container 630 and the monitor 620 may expose the sensing surface 624 of the monitor 620 (e.g., where the sensing surface 624 and/or one or more bottom or side regions of the monitor 620 is protruding from the container 630) such that the sensing surface 624 can contact a wearer’s skin when the container 630 is coupled to the garment 602 being worn. In certain aspects, the physiological monitor 620 protrudes a predetermined distance (e.g., 0.5 mm or more) beyond the first side 631 of the container. This z-height differential may ensure that the sensing surface 624 is the primary point of contact with the skin, minimizing air gaps that could degrade optical sensing performance. A second arrow 603 indicating a rotation of the container 630 and the monitor 620 to show the reverse side of the container 630, i.e., pointing from the connection side to the sensing side of the container 630 holding the monitor 620.

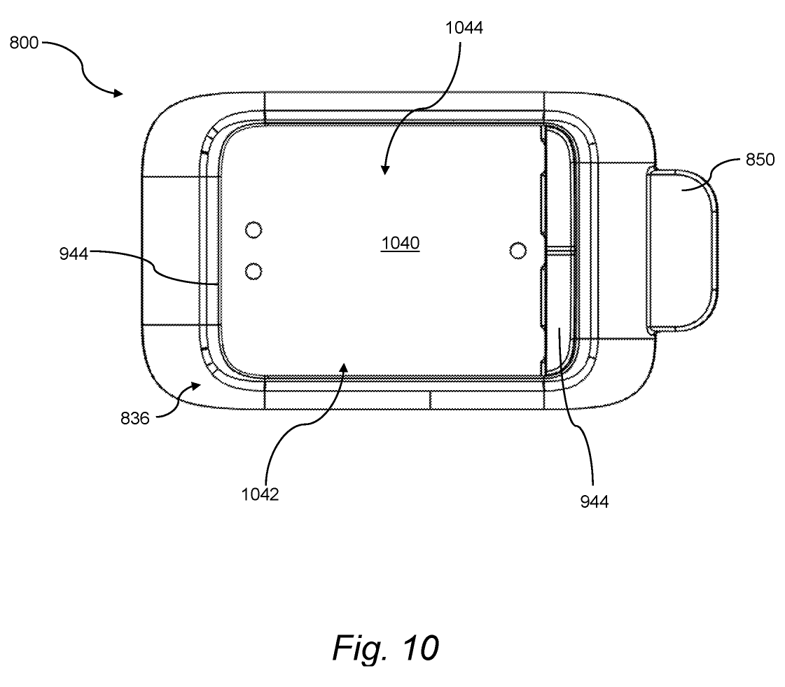

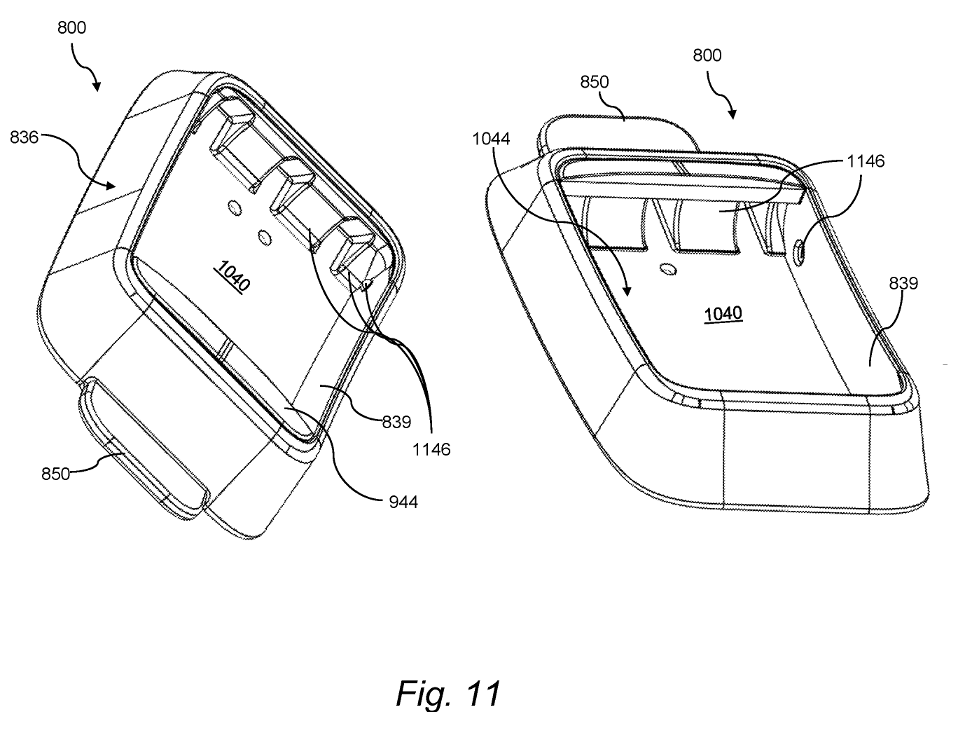

Thus, in an aspect, a system 600 described herein may include: a physiological monitor 620 including one or more sensors 622 on a sensing surface 624 thereof and an RFID reader 626; a garment 602 with an RFID tag 612, a first fastener 614 over the RFID tag 612, and an alignment guide 616 around the first fastener 614; and a container 630 for removably and replaceably attaching the physiological monitor 620. The container 630 may include a rigid skeleton (not viewable in FIG. 6, but shown and described below with reference to FIGS. 10 and 11) and a deformable shell 636 overmolded onto the rigid skeleton. The deformable shell 636 may include: an interior space shaped and sized to receive the physiological monitor 620; an opening to the interior space on a first side 631 of the container 630, the opening positioned to expose the one or more sensors 622 through the opening when the physiological monitor 620 is placed for use in the interior space; and a second fastener 632 on a second side 633 of the container 630, the second side 633 of the container 630 shaped and sized to fit within the alignment guide 616 of the garment 602 in a predetermined position and orientation when the first fastener 614 is coupled to the second fastener 632. The predetermined position and orientation between the alignment guide 616 and the second side 633 of the container 630 may orient the RFID reader 626 of the physiological monitor 620 to retrieve data from the RFID tag 612 of the garment 602. This may be a single, specific position and orientation, or a number of positions and/or orientations in which data can be extracted from the RFID tag 612 by the RFID reader 626.

Thus, in certain aspects, the container 630 may include a skeleton made of a rigid material, such as polycarbonate or ABS, that provides structural support and stability to the container 630. The deformable shell 636 may be overmolded onto the skeleton, and may be made of a flexible material, such as silicone or thermoplastic elastomer. The second fastener 632 (e.g., a hook and loop fastener) may be disposed on the second side 633 of the deformable shell 636, and may be used to secure the container 630 to the garment 602. The deformable shell 636 may define an interior space that is shaped and sized to receive the monitor 620, and may include an opening on the first side 631 that allows for insertion and removal of the monitor 620.

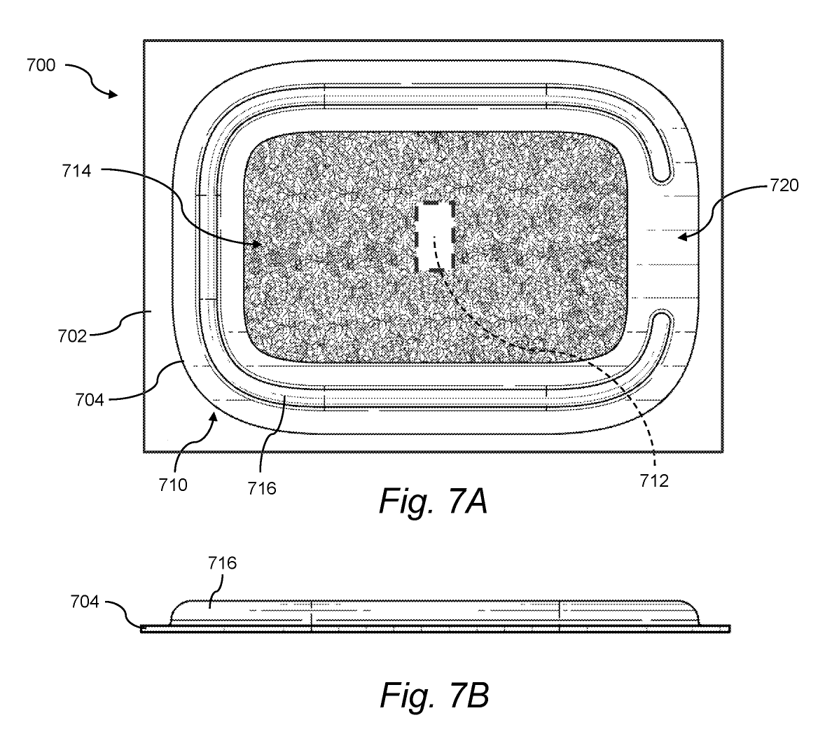

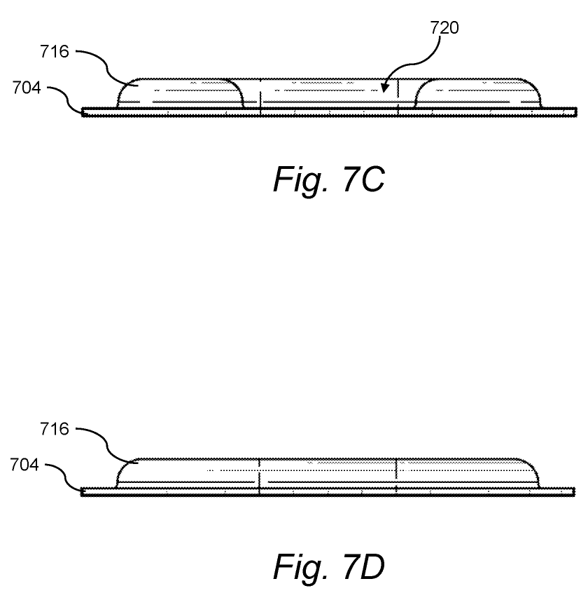

FIG. 7A shows a top view of an attachment region of a wearable article, FIG. 7B shows a side view of an attachment region of a wearable article, FIG. 7C shows a front view of an attachment region of a wearable article, and FIG. 7D shows a rear view of an attachment region of a wearable article. It will be understood that the wearable article 700 that includes the attachment region 710 shown in these figures may be a garment such as the garment 602 described above with respect to FIG. 6, and thus the attachment region 710 may include any of the features described above with reference to FIG. 6, and vice-versa. By way of example, the wearable article 700 may include at least one of a shirt, pants, a compression sleeve, an undergarment, and the like. The wearable article 700 may generally include a sheet of material 702 forming at least a portion of the wearable article 700, a data tag 712 (e.g., an RFID tag), a first fastener 714, and an alignment guide.

In some aspects, the wearable article 700 includes a plurality of attachment regions 710 disposed at different locations on the wearable article 700. Each attachment region 710 may include a respective data tag 712, a respective first fastener 714 (e.g., a hook-and-loop fastener) disposed over the respective data tag 712, and a respective alignment guide (e.g., a raised border 716) disposed at least partially around the respective first fastener 714. In certain aspects, the attachment region(s) 710, including the alignment guide, are disposed on an interior surface of the wearable article 700 such that, when the wearable article 700 is worn, the attachment region(s) 710 face the wearer’s skin to position a coupled container/monitor for physiological sensing.

The sheet of material 702 that forms at least a portion of the wearable article 700 may be formed of one or more materials commonly used in clothing, and more particularly, athletic wear or activewear. This may include, for example, one or more of polyester, spandex/elastane, nylon, wool, bamboo fiber, polypropylene, and the like. In some implementations—e.g., where the attachment region 710 is attachable, permanently or temporarily, to a wearable article 700—the sheet of material 702 may include a fabric patch, an iron-on patch, a taped patch, or the like. It will be understood that, although the sheet of material 702 is shown outside of the perimeter structure 704 of the attachment region 710, in some aspects, the sheet of material 702 forms the perimeter structure 704 of the attachment region 710.

The data tag 712 may be any as described herein—e.g., the data tag 712 may be a radio frequency identification (RFID) tag disposed on, under, or within the sheet of material 702 or another portion of the wearable article 700 and/or attachment region 710. In general, the data tag 712 may be disposed in a location where, when the attachment region 710 receives a container holding a physiological monitor in a predetermined manner and the container is placed for use relative to the attachment region 710 (e.g., in a predetermined position and/or orientation), the data tag 712 can communicate with a data reader disposed on or within the physiological monitor (and/or the container holding the monitor). Although a particular location is shown in FIG. 7A for the data tag 712, other locations along the attachment region 710 (or otherwise along the wearable article 700) are also or instead possible.

The data tag 712 may encode identifying information for the wearable article 700. This information may include, for example, a location of the attachment region 710 relative to the wearable article 700 (e.g., information that the location of the attachment region 710 is disposed along the glute portion of a pair of shorts), a location of the attachment region 710 relative to a body of the wearer (when placed for use), a type of the wearable article 700 (e.g., a SKU), a clothing category (e.g., shorts, pants, shirt, underwear, bra, undershirt, boxers, sock, and so forth), a material type or other characteristic of the wearable article 700, a size of the wearable article 700, preferred sensing operations for the wearable article 700 (or for the specific location along the wearable article 700), and so forth.

In implementations having multiple attachment regions 710, the data tag 712 of each attachment region 710 may encode a unique location identifier corresponding to that attachment region 710. For example, a first attachment region 710 may encode a first location identifier associated with a first body location (e.g., left bicep), while a second attachment region 710 may encode a second, different location identifier associated with a second body location (e.g., right bicep). In this manner, when a container/monitor is coupled to a selected attachment region 710, the monitor may read the unique location identifier to determine which attachment region 710 (and/or which corresponding body location) is in use.

The first fastener 714 may be any as described herein—e.g., the first fastener 714 may include a hook and loop fastener, or more specifically, the hook surface or the loop surface of the hook and loop fastener. The first fastener 714 may be disposed over the data tag 712. In an aspect, the data tag 712 is disposed between the sheet of material 702 and the first fastener 714 such that the first fastener 714 overlays the data tag 712 and the data tag 712 is retained against the sheet of material 702. For example, the data tag 712 may be stitched, laminated, heat-bonded, adhesively bonded, or otherwise captured between the sheet of material 702 and a backing layer of the first fastener 714 (e.g., between fabric and a hook-and-loop patch). In other aspects, other components of the attachment region 710 or wearable article 700 are disposed over the data tag 712, e.g., the sheet of material 702. The first fastener 714 may be configured to engage with a second fastener disposed on a container for holding a physiological monitor. In some aspects, positioning the data tag 712 directly under the fastener minimizes the separation distance between the tag and a corresponding reader on the monitor/container when coupled, improving data exchange reliability without requiring increased reader power. Also or instead, positioning the data tag 712 between the sheet of material 702 and the first fastener 714 may provide mechanical protection for the data tag 712, shielding the data tag 712 from abrasion, bending fatigue, and delamination during repeated attach/detach cycles and laundering of the wearable article 700. In some implementations, a heat-activated adhesive may be used to further secure the data tag 712 and to laminate the first fastener 714 to the underlying sheet of material 702, thereby enhancing the durability and reliability of the attachment region 710.

The alignment guide may include a raised border 716, which may be disposed at least partially around the first fastener 714. In some aspects, the raised border 716 is formed of a material that is more rigid than the sheet of material 702 so that the raised border 716 maintains its shape as a mechanical key during donning, doffing, and physical activity. By way of example, the raised border 716 may be formed from a molded polymer (e.g., TPU or similar), a silicone/elastomer having a higher durometer than the surrounding sheet of material 702, a composite structure, or the like, and may be bonded, stitched, heat-bonded, or otherwise secured to the sheet of material 702. The raised border 716 may include one or more features providing a mechanical key to enforce a predetermined position and orientation of a device with a corresponding shape placed thereon. For example, the raised border 716 may define a physical barrier that abuts the sidewalls of a container as described herein. This interference may inhibit or prevent lateral shear movement and/or rotation of such a container relative to a garment during high-intensity physical activity, encouraging the sensor(s) of a monitoring device to remain fixed over the target tissue. More specifically, the raised border 716 may include one or more features to enforce a specific position and orientation of a container for a monitor. In certain aspects, the raised border 716 may include a break 720 or other discontinuity, change in shape, or the like, that acts as a feature to position and/or orient a container when placed for use in the alignment guide. For example, the raised border 716 of the alignment guide may form an open C with an opening (e.g., the break 720) or other shape providing the mechanical key to position and align a container for use with an underlying tag and/or to position the sensor(s) of the monitor at a known location on a user’s body. The alignment guide, which may include the raised border 716 and/or the break 720, may thus serve as a tactile and/or visual guide for a user to properly position and orient the a container and sensor(s) for use relative to the attachment region 710, and as a result, with a location on a user’s body.

The break 720 in the raised border 716 may provide several technical advantages. In certain aspects, the break 720 cooperates with a tab on the container to create a single valid insertion orientation, thereby enforcing rotational alignment and ensuring that an RFID reader of the physiological monitor is positioned correctly relative to the RFID tag for reliable data exchange. The open-C shape or similar discontinuity may also or instead provide a tactile reference that allows a user to orient the container by feel alone, without visual inspection, which may be beneficial for attachment regions disposed on the interior of garments that are not easily visible during donning. In some aspects, the raised border 716 may be formed in a different color than the surrounding sheet of material 702 to further visually distinguish the alignment guide and indicate how the container needs to be keyed relative to the first fastener 714.

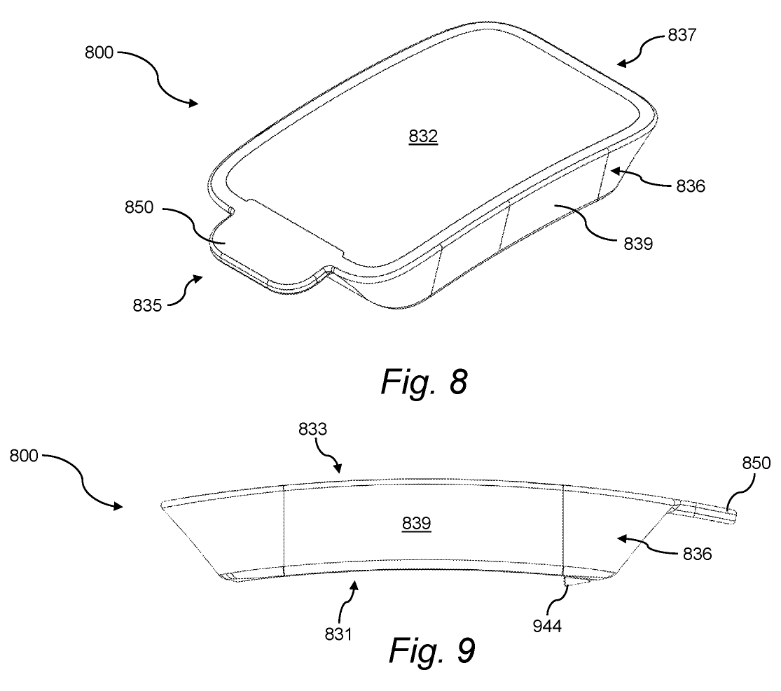

FIG. 8 shows an isometric view of a container for a physiological monitor, FIG. 9 shows a side view of a container for a physiological monitor, FIG. 10 shows a bottom view of a container for a physiological monitor, and FIG. 11 shows perspective bottom views of a container for a physiological monitor. The container 800 may be the same or similar to any as described herein, such as that described with reference to any of FIGS. 6–7D. The container 800 may generally be configured for holding a physiological monitoring device in a manner such that, when the container 800 is coupled with an attachment region of a garment (such as any as described herein) and the garment is placed for use on a wearer’s body, the physiological monitoring device is positioned for sensing one or more predetermined physiological signals, such as any as described herein. In one aspect, the physiological monitoring device may project from the container 800 such that a sensing surface is exposed and/or one or more sensors have a communication path (e.g., a relatively unobstructed line of sight for optical sensors, an electrical connection for electrical sensors, a relatively unobstructed path for detecting thermal radiation of temperature sensors, and the like) to a sensing surface, such as skin of a wearer of a garment coupled to the container 800. The container 800 may define a first side 831, a second side 833, a front side 835, and a back side 837. The first side 831 may define an opening 1042 for receiving and selectively retaining a physiological monitoring device. In embodiments, the first side 831 may contact or face a wearer’s body when a garment coupled to the container 800 is placed for use on the wearer. The second side 833 may define an engagement surface structurally configured for engaging with an attachment region of a wearable article as described herein, and may face away from a wearer’s body when a garment coupled to the container 800 is placed for use on the wearer. The container 800 may generally include a skeleton 1040 and a deformable shell 836 enveloping at least a portion of the skeleton 1040.

For example, the container 800 may include a skeleton 1040 of substantially rigid material—e.g., a material that is more rigid than the deformable shell 836. By way of example, the rigid material of the skeleton 1040 may include polycarbonate or the like. The skeleton 1040 may also or instead include acrylonitrile butadiene styrene (ABS), acrylic, polybutylene terephthalate (PBT), styrene ethylene butadiene styrene (SEBS), combinations thereof, and the like. In embodiments utilizing RFID or similar wireless communication between the monitor and the garment, the material of the skeleton 1040 (and the insert described herein, where present) may be selected to be substantially radio-frequency transparent at the operating frequencies of a tag and reader, thereby avoiding or reducing signal attenuation between the sensing surface and the attachment region. In such embodiments, the material of the deformable shell 836 may also be selected to be substantially radio-frequency transparent at the operating frequencies of the tag and reader (e.g., materials such as silicone, TPE, EPDM, rubber, or other elastomers), and may be selected and/or formulated to avoid constituents (e.g., metal-filled additives) that would materially attenuate communication between the RFID tag and the RFID reader. In general, the skeleton 1040 may provide rigidity for the container 800, and/or a structure that can be overmolded with a softer material to form the general structure of the container 800. In an example, the skeleton 1040 provides a substantially rigid backbone structure for the container 800, e.g., along the second side 833 of the container 800, while permitting softer regions of the overmolded structure to flex and stretch to accommodate insertion and removal of a sensor module therein. As shown in the figures, the second side 833 of the container 800 may include a predetermined curvature. The predetermined curvature may be selected for comfort of a wearer of a garment when the container 800 is engaged therewith (e.g., where the curvature is selected to correspond to common human body curvature in one or more select areas). A radius of curvature for the container 800 may also or instead be used to control a normal force toward a wearer’s skin when the container 800 is engaged to a garment or similar, or otherwise control an engagement between the sensing surface of a physiological monitoring device and skin of a wearer. The skeleton 1040 may be longer than a length of the physiological monitoring device, e.g., so that the skeleton 1040 extends beyond the length of the device and the opening for the device—that is, horizontally in FIG. 10 beyond the edges of the opening 1042—so that the relatively rigid skeleton 1040 can extend the structural contact surface with a garment beyond the extent of the monitor, thus increasing or otherwise controlling the normal force that is generated by the garment against a user’s skin. Thus, the skeleton may generally be wider than the opening 1042 along at least one axis. In some aspects, the skeleton 1040 may be longer than a length of the opening 1042 of the container 800, and/or wider than a width of the opening 1042 of the container 800.

The container 800 may include a deformable shell 836 overmolded onto the skeleton 1040, or otherwise enveloping at least a portion of the skeleton 1040. In some aspects, the deformable shell 836 may cover the entirety of the skeleton 1040. Thus, while the skeleton 1040 is visible in, e.g., FIG. 10, the skeleton 1040 may be completely overmolded and not externally visible. In other aspects, the deformable shell 836 may cover a majority of the skeleton 1040, or less than a majority thereof. In an example, the deformable shell 836 may be overmolded onto the skeleton 1040 such that a bottom portion of the skeleton 1040 is exposed within an interior space 1044 of the container 800. The deformable shell 836 may have a first side and a second side, e.g., where these sides correspond directly to the first side 831 and the second side 833 of the container 800 as a whole.

The deformable shell 836 may be made from a material that is more pliable than that of the skeleton 1040, e.g., with a lower bulk modulus or elastic modulus. For example, the deformable shell 836 may be formed of silicone or similar. The deformable shell 836 may also or instead be formed at least in part using one or more of the following: organic semiconductors (e.g., fullerenes and carbon nanotubes (CNTs)), other polymers, a thermoplastic elastomer (e.g., thermoplastic vulcanizates (TPVs)), ethylene propylene diene monomer (EPDM), rubber, and the like. In some aspects, the deformable shell 836 has a predetermined tackiness (e.g., selected surface friction and/or surface finish) that resists lateral movement (e.g., sliding and/or rotation) of the container 800 relative to the wearer’s skin when placed for use. For example, the deformable shell 836 may be formed from a silicone elastomer having a selected durometer and surface finish, may include a tacky coating, may include micro-texturing, and/or may otherwise be configured to increase friction at the skin interface.

The deformable shell 836 may have a predetermined thickness along one or more portions of the container 800. For example, the deformable shell 836 may have a thickness along the second side 833 of the container 800—e.g., between the interior space 1044 and the second side of the deformable shell 836—not greater than a sensing distance for an RFID tag to be sensed by a physiological monitor when placed for use in the interior space 1044 of the container 800.

The deformable shell 836 may define an interior space 1044 shaped and sized to retain a physiological monitor, where the interior space 1044 is accessible through an opening 1042 in the structure of the container 800. Thus, the interior space 1044 may include an opening 1042 on the first side 831 of the deformable shell 836 for removal of the physiological monitor from, and replacement of the physiological monitor to, the interior space 1044.

The deformable shell 836 may cover a portion of the skeleton 1040, which may form a backbone structure (e.g., a substantially inflexible backbone structure) for the container 800. The deformable shell 836 may project from this backbone structure toward the first side 831 of the container 800, forming sidewalls 839 for the container 800 (or at least a portion thereof). In some cases, the deformable shell 836 may extend downward toward the first side 831 of the container 800 such that the deformable shell 836 forms the perimeter (rim) of the opening 1042 (wholly or partially), or an interior wall around the interior space 1044. This may be advantageous, as the rim of the opening 1042, any extensions 944 therefrom, and/or portions of the sidewalls 839 may contact a wearer’s skin when the container 800 is engaged with a wearable article. Where the deformable shell 836 includes a pliable, substantially elastic material, this may promote comfort for the wearer. Also or instead, the material of the deformable shell 836 (e.g., the pliability thereof) may be selected to provide a predetermined tackiness for the deformable shell 836 that resists lateral movement of the container 800 relative to skin of the wearer, which may be advantageous for comfort, stability, and/or quality of sensing operations and sensed data. In some aspects, the container 800—e.g., at least a portion thereof such as the deformable shell 836—may include a silicone paint or similar that is applied wholly or partially thereon. For example, such a silicone paint may be applied to all external surfaces, which may serve an aesthetic purpose (e.g., to prevent lint from building up) and/or a functional purpose (e.g., to allow for a smooth sensation on the skin). In some aspects, preventing lint and debris accumulation on the deformable shell 836 may help preserve the shell’s predetermined tackiness at the skin interface, which may be useful for maintaining positional stability of the container 800 (and thus sensor alignment) during physical activity. Additionally, the smooth coating may reduce friction against adjacent garment fabric layers, allowing the garment to slide naturally over the container 800 without bunching or snagging, which could otherwise create pressure hotspots or displace the container 800.

In certain implementations, retention of the physiological monitor is configured such that the physiological monitor projects at least partially from the opening 1042 when fully seated within the container 800. In this manner, the depth of the interior space 1044 may be selected such that it is shorter than a height of the physiological monitor. The retention of the physiological monitor may be any as described herein, including one or more of a friction fit, snap fit, mechanical keying, and the like.

In some aspects, the container 800 includes one or more extensions 944 that extend from a body of the container 800 over at least a portion of the opening 1042. An extension 944 may be part of the deformable shell 836 in some aspects. This may be advantageous in implementations where the deformable shell 836 has a pliability that permits deformation of the extension 944 for inserting the physiological monitor within the interior space 1044—e.g., where the extension 944 deforms to allow the physiological monitor to pass through the opening 1042, and where the extension 944, through an elasticity of the material of the deformable shell 836, then elastically generates forces returning to an at-rest shape in order to securely conform around the physiological monitor. In this manner, one or more extensions 944 may also or instead be used to promote secure retention of the physiological monitor within the container 800. In some aspects, one or more extensions 944 may be deformable to assist in removing the physiological monitor from the container 800, e.g., by pulling back a pliable extension 944 to permit removal of the monitor. In other aspects, one or more of the extensions 944 may be part of the skeleton 1040 or otherwise be formed of a more rigid material than the deformable shell 836.

In an aspect, the skeleton 1040 may extend laterally across the entire length of the interior space 1044, e.g., forming an entire base thereof. Moreover, the skeleton 1040 may be longer than a length of the interior space 1044, and/or a length of the opening 1042. For example, in an aspect, the skeleton 1040 extends beyond the interior space 1044 of the deformable shell 836 in at least one dimension, thereby providing a rigid structure for the container 800 extending beyond the physiological monitor in the at least one dimension when the physiological monitor is placed for use in the interior space 1044 of the container 800. This may be a longest of three orthogonal dimensions passing through a point in the interior space 1044—e.g., the longest dimension of the interior space 1044 or the physiological monitor, or along a horizontal axis in FIGS. 9 or 10. Such an elongated skeleton 1040 may be useful for providing, in combination with a garment or the like, a desired normal force urging a physiological monitor toward a sensing surface when in use. For example, the combination of the rigid skeleton 1040 (which may be formed via the first shot of material in an injection molding operation or similar), the deformable shell 836 (which may be formed via the second shot of material in an injection molding operation or similar), and the protrusion of the monitor from the container 800 may provide advantageous sensor coupling. This enhanced coupling may represent an improvement over other arrangements, such as where a physiological monitor is backed by fabric and/or other non-rigid materials, which may yield worse signal to noise ratios than the present teachings.

The second side 833 of the deformable shell 836—or more generally, the second side of the container 800—may have a fastener disposed thereon (i.e., a second fastener 832 as described herein, which may be structurally configured to couple with a first fastener on a wearable article). In an example, this second fastener 832 may include a hook and loop fastener.

As described above, the second side 833 of the container 800 may include a predetermined curvature. Thus, in some aspects, the second side 833 of the deformable shell 836 may have a curvature with a center of curvature in a location toward the first side 831 of the deformable shell 836 from the second side 833 of the deformable shell 836. The curvature of the second side 833 may be selected to more closely align with the curvature of the garment when worn on the body, thereby improving wearer comfort. A curvature that is too flat or too pronounced may result in reduced comfort during extended wear. Additionally, the curvature may be selected to accommodate positioning of the container 800 at numerous body locations where a user may wear the container 800, providing a balance that is neither too round nor too flat to enable versatile placement across different anatomical regions. The curvature of the second side 833 may also or instead assist in retaining the position of the physiological monitor’s sensors relative to the skin of the wearer, promoting consistent sensor-to-skin contact during use and thereby supporting accurate physiological data acquisition. That is, the curvature may bend toward the sensing side of a monitor and away from a garment-facing side of the monitor, with the center for the corresponding radius of curvature conceptually positioned at or below the skin surface of the user when the monitor is placed for use.

As described above, the physiological monitoring device, when inserted within the container 800, may project from the container 800 such that a sensing surface is exposed and/or one or more sensors have a communication path to a sensing surface, such as skin of a wearer of a wearable article that couples with the container 800. That is, the opening 1042 may be positioned to expose one or more sensors of the physiological monitor through the opening 1042 for physiological sensing when the physiological monitor is placed for use in the interior space 1044 of the container 800 and the container 800 is placed for use on a user (e.g., on a wearable article being worn by the user).

The container 800 may include a tab 850, e.g., projecting from the front side 835 of the container 800. For example, the container 800 may include a tab 850 extending from a sidewall 839 of the deformable shell 836, or otherwise from a sidewall 839 of the structure of the container 800 (e.g., a portion of a sidewall 839 that also, at least in part, surrounds the interior space 1044). The tab 850 may provide an alignment key for enforcing a predetermined orientation of the container 800 in a guide (e.g., an alignment guide of an attachment region of a wearable article as described herein). The tab 850 may also or instead provide an ergonomic feature to assist a user with placement and/or removal of the container 800 from a wearable article, and/or placement and/or removal of a physiological monitor from the container 800 (e.g., by providing a region intended to be held by a user during one or more of these actions). The tab 850 may also or instead provide a visual guide for a user, e.g., to align the second side 833 of the container 800 with an attachment region of a wearable article, which may include a cooperating feature that is visually identifiable (e.g., the void in a c-shaped perimeter that is configured to align with the tab 850). Structurally, the tab 850 may nest within a break of a raised border on a wearable article and/or attachment feature thereon as described herein. This nesting may create a mechanical interlock that further restricts rotation of the container 800 relative to an attachment region of a wearable article. In one aspect, the tab 850 may advantageously be positioned on the garment side of the container 800 so that the container 800 can create a void space for the tab 850 when a garment with the container 800 is placed for use against the skin, thereby reducing potential discomfort from the resulting protrusion in the surface of the container 800.

As shown in FIG. 11, the container 800 may further include one or more engagement features 1146 disposed in the interior space 1044 thereof. The engagement features 1146 may aid in aligning, protecting, and/or securing a physiological monitor within the container 800. In some aspects, the engagement features 1146 may ensure repeatable, precise positioning of the physiological monitor within the container 800 across multiple insertion and removal cycles, which may maintain consistent sensor-to-skin alignment and avoid degradation of sensing accuracy that could result from rotational or translational misalignment of the physiological monitor within the container 800. The form-fitting interior defined at least in part by the engagement features 1146 may visually and tactilely convey to a user the correct insertion orientation. For example, if a user were to press the physiological monitor into the container 800 in an incorrect position, the sidewalls 839 may bulge out and appear or feel incorrect, whereas in the correct position, the physiological monitor may snap into the interior with minimal gaps. In some aspects, the engagement features 1146 and other internal surfaces of the container 800, such as the sidewalls 839, may mechanically constrain the position of the physiological monitor, which may prevent the physiological monitor from shifting within the container 800 during high-intensity physical activity and may reduce intermittent loss of sensor contact or motion artifacts in sensed data. For example, an engagement feature 1146 may conform to an exterior shape of a physiological monitor. Also or instead, an engagement feature 1146 may engage with—e.g., mechanically key with—one or more features of the physiological monitor. Also or instead, an engagement feature 1146 may form all or a portion of a snap-fit connection and/or a friction fit connection with the physiological monitor. Also or instead, an engagement feature 1146 may include one or more of a projection or an indentation that cooperates with a corresponding structural element on the physiological monitor. An engagement feature 1146 may be made from the same or similar materials as, and may be part of, one or more of the skeleton 1040 and the deformable shell 836. By way of example, the one or more engagement features 1146 may each be formed with (e.g., using the same mold) the same material as the deformable shell 836 so that the deformable shell 836 can deform around and elastically retain a monitor positioned therein.

It will be understood that the container 800 may also or instead include further functionality and components configured to provide this functionality. By way of example, in an aspect, the skeleton 1040 may define, include, or otherwise be coupled with a charging component, e.g., a wireless charger disposed along a backbone structure of the container 800. In this manner, when a physiological monitor is inserted into the container 800, a charging operation for the physiological monitor may occur. To this end, the container 800 may include a charging port for charging a battery / charging component within the container 800, for subsequently inserting another device therein for a wireless charging operation or similar. This or other functionality may also or instead be provided by components on or within the attachment region of a wearable article. By way of further example, the charging component may include an inductive charging coil (and associated power management circuitry) disposed within or coupled to the skeleton 1040 and positioned such that, when the physiological monitor is placed for use in the interior space 1044, a corresponding charging coil of the physiological monitor is aligned (or sufficiently aligned) for inductive power transfer. In some aspects, the charging component is configured to operate according to a wireless charging standard and/or includes alignment features (mechanical and/or magnetic) that promote repeatable positioning of the physiological monitor relative to the charging component. In certain aspects, the container includes a battery and/or electrical contacts/port (e.g., USB) for charging the battery or otherwise powering the charging component. Other functionality is also or instead possible.

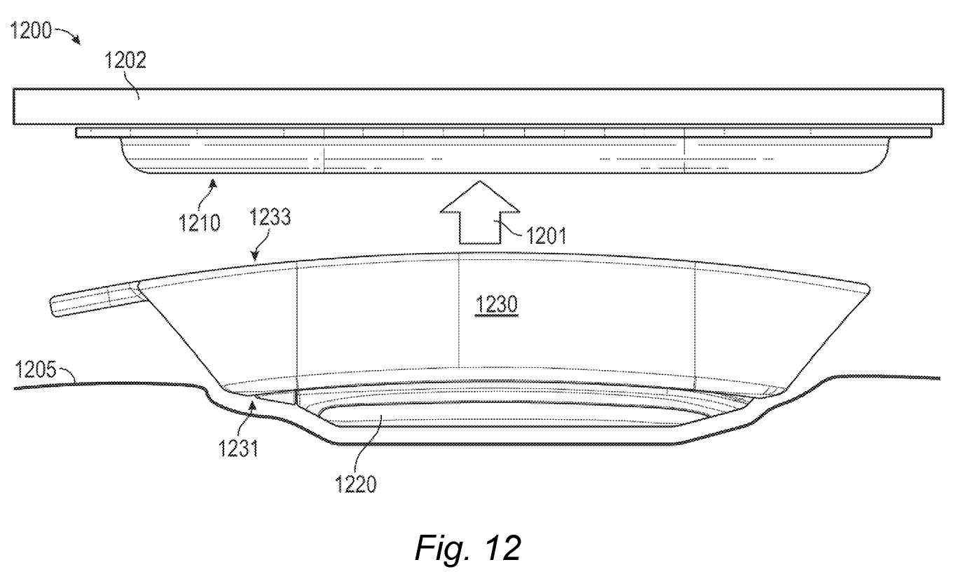

FIG. 12 shows a system for physiological monitoring. The system 1200 may include any of the features of other systems described herein, e.g., with reference to FIG. 6, but where this figure shows a representation of the system 1200 in use. In particular, the system shows a wearable article 1202 with an attachment region 1210, a container 1230 holding a physiological monitor 1220 on a first side 1231 thereof, and skin 1205 of a wearer of the wearable article 1202. For convenience and understanding, the attachment region 1210 and the container 1230 are shown separated; however, it will be understood that, as represented by the arrow 1201, the container 1230 would be coupled with the attachment region 1210, and the attachment region 1210 (and the wearable article 1202) would generally conform (e.g., flex or bend) to the second side 1233 of the container 1230 when in use.

In an aspect, the wearable article 1202 and/or the attachment region 1210 is a substantially elastic material that is capable of conforming to the shape of the container 1230 and/or of applying a force directing the container 1230 (and thus the physiological monitor 1220) into the skin 1205 of the wearer. That is, the tension provided by the wearable article 1202 and/or the attachment region 1210 may create a normal force pressing the container 1230 and thus the physiological monitor 1220 against the skin 1205 of the wearer. This normal force may be the result of hoop stress (circumferential stress) created by the wearable article 1202 urging the container 1230 (e.g., an elongated rigid backbone thereof, which may include a curved profile structurally configured to create a predetermined hoop stress) toward skin 1205 of the wearer. More generally, a rigid skeleton of the container 1230 may act as a cantilevered spring or flexure. In this manner, when the wearable article 1202 is tightened, the curved ends of the container 1230 may be pulled downward, generating a biasing force that leverages the center of the container 1230 (and the physiological monitor 1220 held therein) into the skin 1205 to improve signal quality. And in certain aspects, the combination of the container 1230 and the physiological monitor 1220 may deform the skin by creating an indent into tissue of a wearer while wearing the wearable article 1202. This arrangement may promote greater optical or electrical coupling of the sensor(s) of the monitor with the target tissue for measurement.

The use of a rigid skeleton within a low durometer material can provide significant advantages in this context. For example, the rigid skeleton may provide a rigid backing structure for the hook and loop fastener (or other fastener) disposed on the second side 1233 of the container 1230. In some aspects, this rigid backing structure may help the fastener surface maintain a preferred shape for engagement with a complementary fastener on the wearable article 1202, rather than deforming under external forces that might otherwise compromise the fastener connection. For example, without a rigid backing, the fastener surface may tend to flex, curl, bend, wrinkle, or otherwise deform during physical activity by the wearer, potentially reducing the contact area or causing physical displacement between mating fastener surfaces and weakening the attachment. By providing a substantially rigid substrate beneath the fastener, the rigid skeleton may help ensure that the fastener surface remains relatively flat or maintains a desired curvature, thereby promoting consistent and reliable engagement with the complementary fastener on the wearable article 1202. The rigid skeleton can also or instead be used to increase a normal force generated by the hoop tension in an elastic material that secures the physiological monitor 1220 to a user’s skin. For example, the rigid skeleton can extend surface area or radial dimensions beyond the exterior of the physiological monitor 1220 to control and increase normal forces. For example, by extending a first radial length of the exoskeleton beyond a second radial length of the physiological monitor 1220, more particularly along an axis tangent to the directrix (or circular cross section) of a cylinder containing the curved surface of the container and perpendicular to a second axis of the cylinder, a normal force created by the tension of a fitted strap or garment can be increased, thereby improving the functional engagement of contact-based sensors (e.g., optical sensors) with the skin of a wearer.

Further, portions of the container 1230 on the first side 1231 thereof that contact the skin 1205 of the wearer may be structurally configured to prevent lateral movement along the skin 1205, which can promote improved sensing. For example, if these portions are formed of a silicone based material or the like, the tackiness of this material may stabilize the relative position of the container 1230 (and thus the physiological monitor 1220) relative to the skin 1205.

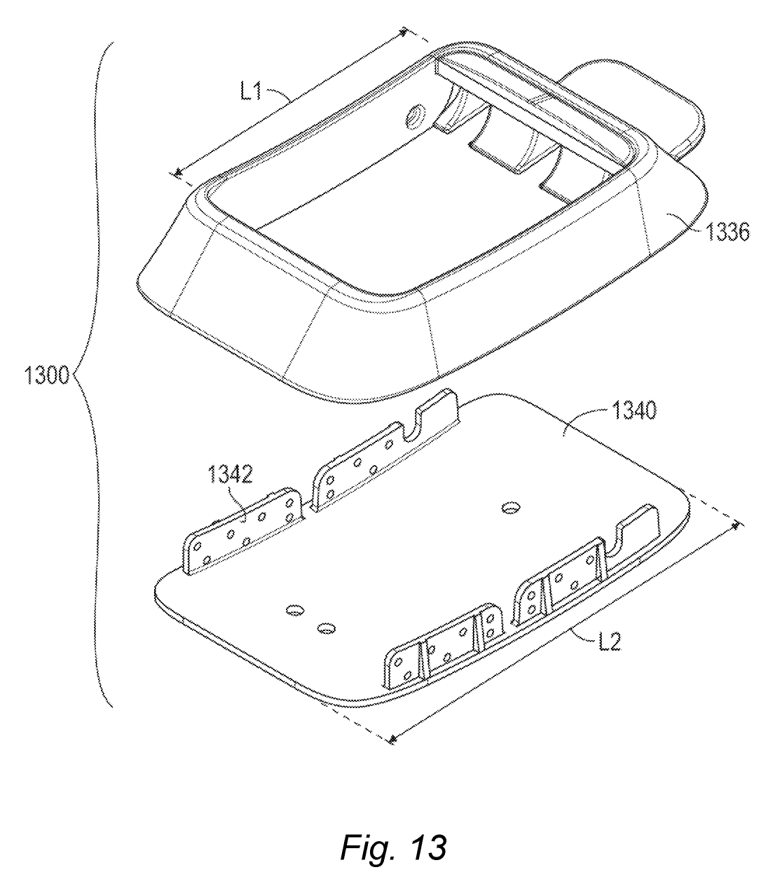

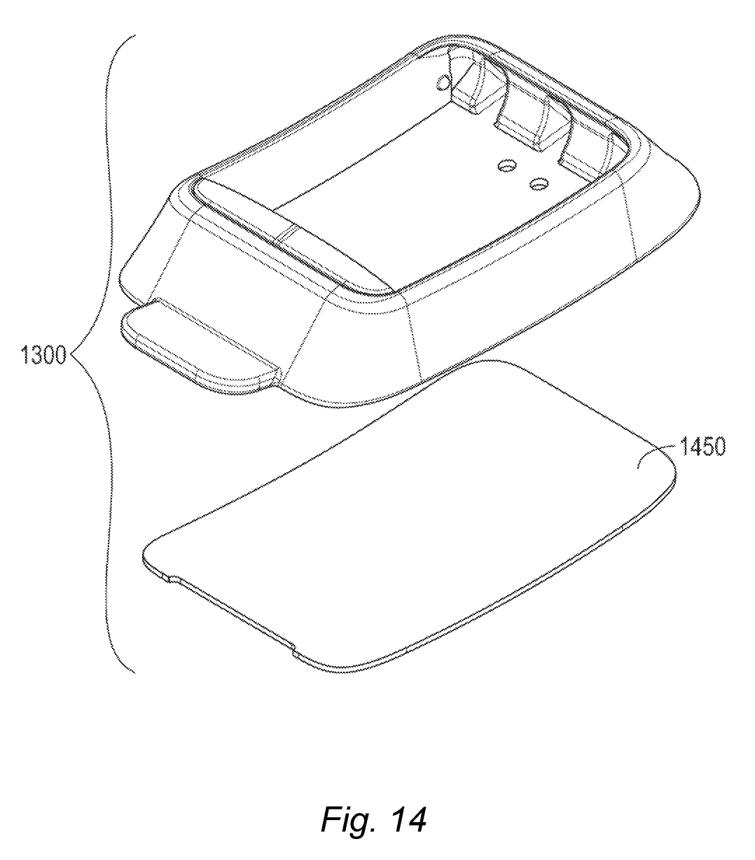

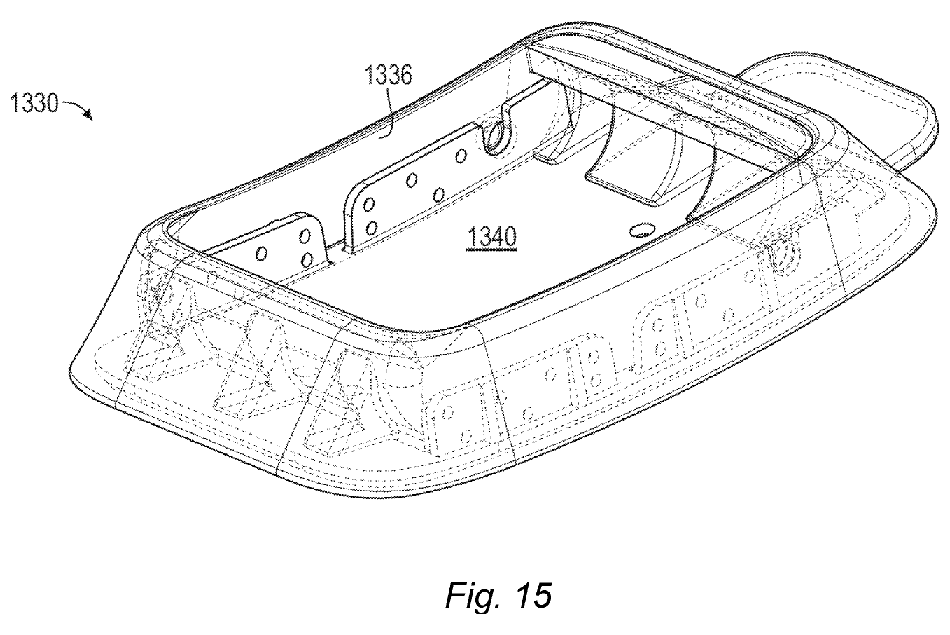

FIGS. 13 and 14 shows portions of a container for a physiological monitor, and FIG. 15 shows a container with a semi-translucent shell for context and understanding of certain aspects of the present teachings. Specifically, these figures are provided by way of example to show an arrangement of a container 1300 for a physiological monitor that includes a skeleton 1340 of rigid material, a deformable shell 1336, and an insert 1450.

The skeleton 1340 may be any as described herein. In some aspects, the skeleton 1340 includes an elongated body that can form the backbone of the container 1300. As described herein, the skeleton 1340 may extend beyond the interior space of the deformable shell 1336 in at least one dimension, thereby providing a rigid structure to the container 1300 extending beyond a physiological monitor placed therein in the at least one dimension when the physiological monitor is placed for use in the interior space of the container 1300. This dimension may be the longest of three orthogonal dimensions passing through a point in the interior space. That is, the dimension may be a length. For example, length L1 (the length of the opening of the deformable shell 1336 configured to receive a physiological monitor) may be shorter than the length L2 of the skeleton 1340, even when accounting for a curved body of the skeleton 1340. The length L2 of the skeleton 1340 and/or the curve of the curved body of the skeleton 1340 may be structurally configured to increase the hoop stress described herein, which may improve sensor coupling between a sensing surface of a physiological monitor and tissue of a wearer of a garment coupled to the container 1300 by increasing the normal force therebetween.

In an aspect, the skeleton 1340 is formed of a material that is more rigid that the deformable shell 1336. The skeleton 1340 may be formed during a manufacturing process such as an injection molding process or similar. For example, the skeleton 1340 may be formed as the first shot of material in an injection molding process or similar, and the deformable shell 1336 may be formed as a subsequent shot (e.g., the second shot) of material (e.g., different, more flexible material than the skeleton 1340). That is, a manufacturing operation for the container 1300 may include overmolding at least a portion of the skeleton 1340 with a substantially deformable material to provide the deformable shell 1336 that at least partially defines an interior space shaped and sized to retain a physiological monitor. Thus, the material of the deformable shell 1336 may adhere to the material of the skeleton 1340, such that the deformable shell 1336 is at least partially overmolded onto the skeleton 1340. In one aspect, the skeleton 1340 is a rigid plastic material and the deformable shell 1336 is silicone or similar.

The skeleton 1340 may include one or more extensions 1342. The extensions 1342 may serve as anchor points that aid in securing the deformable shell 1336 onto the skeleton 1340. Also or instead, the extensions 1342 may provide stability for the structure of the container 1300 and/or may aid in securing and holding a physiological monitor therein.

In some aspects, the extensions 1342 increase the bonding surface area between the rigid skeleton 1340 and the overmolded deformable shell 1336, which may resist delamination of the shell from the skeleton during repeated insertion and removal of the physiological monitor and during physical activity by the wearer. By providing discrete mechanical interlocking points, the extensions 1342 may prevent the deformable shell 1336 from peeling away from the skeleton 1340 at the edges during manufacturing or use. The extensions 1342 may also help maintain dimensional accuracy of the overmolded container 1300 during the manufacturing process by mechanically registering the deformable shell 1336 to the skeleton 1340, which may help ensure that the interior space dimensions and the opening geometry remain within tolerances for proper monitor retention. In certain aspects, one or more of the extensions 1342 may form at least a portion of the geometry of one or more engagement features within the interior space, such as the engagement features 1146 described herein, thereby providing additional mechanical retention for the physiological monitor. The container 1300 may further include an adhesive layer, such as a hot melt adhesive, disposed on a surface of the skeleton 1340 to secure additional components thereto, such as a fastener or an insert as described herein. The adhesive layer may be selected to provide adhesion between materials having different surface energies, such as between a polycarbonate skeleton and a nylon-based fastener material, and may be formulated to withstand exposure to machine wash cycles, perspiration, sebum, cleaning agents, and the like.

The deformable shell 1336 may be any as described herein. For example, the deformable shell 1336 may define an interior space shaped and sized to retain the physiological monitor.

FIG. 14 shows the insert 1450, which may be added to the structure of the container 1300 after the deformable shell 1336 is overmolded onto the skeleton 1340. The insert 1450 may include a fastener as described herein, i.e., on an exterior surface thereof, and may be structurally configured to snap-fit, adhere, and/or otherwise mechanically lock into the skeleton. In this configuration, the skeleton may provide the structural rim for the overmolding of the shell, while the insert 1450 provides a rigid backing plate for the fastener. Thus, in some aspects, the insert 1450 includes a fastener, e.g., on an exterior surface thereof. Also or instead, the insert 1450 may include other components that can offer further functionality for the container 1300, such as a battery / charging component or similar. The insert 1450 may include a substantially rigid structure, where such rigidity (and/or a shape of the insert 1450) may aid in the hoop stress provided by the container 1300 when coupled to the attachment area of a garment worn by a user. In other aspects, the insert 1450 is substantially flexible.

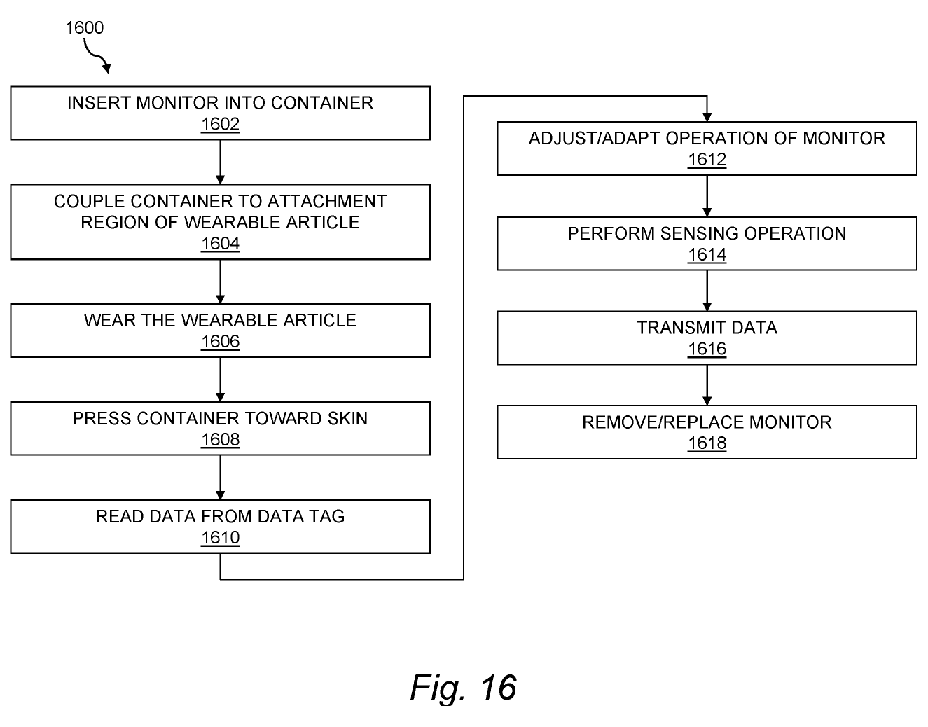

FIG. 16 is a flow chart of a method for coupling a physiological monitor with a wearable article. The method 1600 may be performed using any of the systems, devices, and components described herein, such as those described with reference to FIGS. 6–15 above.

As shown in a step 1602, the method 1600 may include inserting a physiological monitor having one or more sensors on a sensing surface thereof into a container. The container may include a first side and a second side, where the first side defines an opening to an interior space shaped and sized to receive the physiological monitor, and the second side includes a fastener. The container may further include a rigid skeleton disposed at least along the second side of the container to form a backbone structure thereof. A deformable shell may be overmolded onto the skeleton and may define at least a portion of a perimeter of the opening to the interior space. When the physiological monitor is disposed within the container, the sensing surface may be exposed through the opening. In certain aspects, inserting the physiological monitor into the container includes elastically deforming one or more extensions of the container to allow the physiological monitor to pass through the opening. The extensions may elastically return to conform around the physiological monitor to retain the physiological monitor within the container.

As shown in a step 1604, the method 1600 may include coupling the second side of the container to an attachment region of a wearable article. The attachment region may include a complementary fastener to engage with the fastener of the container. The attachment region may further include an alignment guide to provide a predetermined position and orientation of the container when coupled to the attachment region. In certain aspects, coupling the second side of the container to the attachment region of the wearable article includes aligning the container with the alignment guide. The alignment guide may include a raised border having one or more features providing a mechanical key, and aligning the container may include engaging a corresponding feature of the container (such as the tab described herein) with the mechanical key.

As shown in a step 1606, the method 1600 may include wearing the wearable article such that the sensing surface is disposed adjacent to skin of a wearer. This may also or instead include performing a physical activity while wearing the article.

As shown in a step 1608, the method 1600 may include pressing the container toward the skin of the wearer. In certain aspects, the method 1600 includes generating a normal force pressing the container toward the skin of the wearer via hoop stress created by the wearable article. The container and the rigid backbone structure may work together with garment tension to push the sensor into the user’s tissue. The combination of the container height, garment tension, and rigidity of the skeleton (which may be formed of polycarbonate or similar) may collectively increase the pressure between the sensor and the user’s skin compared to configurations without a rigid backbone. In certain aspects, the method 1600 includes creating an indent into tissue of the wearer with the container and the physiological monitor for optical or electrical coupling between one or more sensors and the target tissue. The container design may maximize force in the relevant contact area where the sensor interfaces with skin while minimizing force in superfluous areas, e.g., because the sensor surface protrudes from the container.

The deformable shell may contact the skin of the wearer and may resist lateral movement of the container relative to the skin due to a tackiness of the deformable shell. This tackiness may stabilize the relative position of the container and the physiological monitor during physical activity by the wearer.

As shown in a step 1610, the method 1600 may include reading data from a data tag disposed on the attachment region of the wearable article. The data may be read using a data reader of the physiological monitor, such as an RFID reader as described herein. The data may include at least one of location of the attachment region on the wearable article, location of the attachment region relative to the wearer, information regarding the wearer, information regarding the wearable article, and the like.

As shown in a step 1612, the method 1600 may include adapting operation of the physiological monitor based on the data read from the data tag. In certain aspects, adapting operation includes selecting one or more of a filter, a processing model, or a physiological signal detection algorithm based on the data.

As shown in a step 1614, the method 1600 may include performing a sensing operation using one or more sensors of the physiological monitor. In certain aspects, the sensing operation includes acquiring heart rate data using photoplethysmography. Other sensing operations are also or instead possible.

As shown in a step 1616, the method 1600 may include transmitting data acquired during the sensing operation to a remote processing resource for analysis.

As shown in a step 1618, the method 1600 may include removing the physiological monitor from the container and replacing the physiological monitor to the container. The container design may thus enable the physiological monitor to be removable and replaceable, and may allow the physiological monitor to be positioned at a plurality of different locations on different garments.

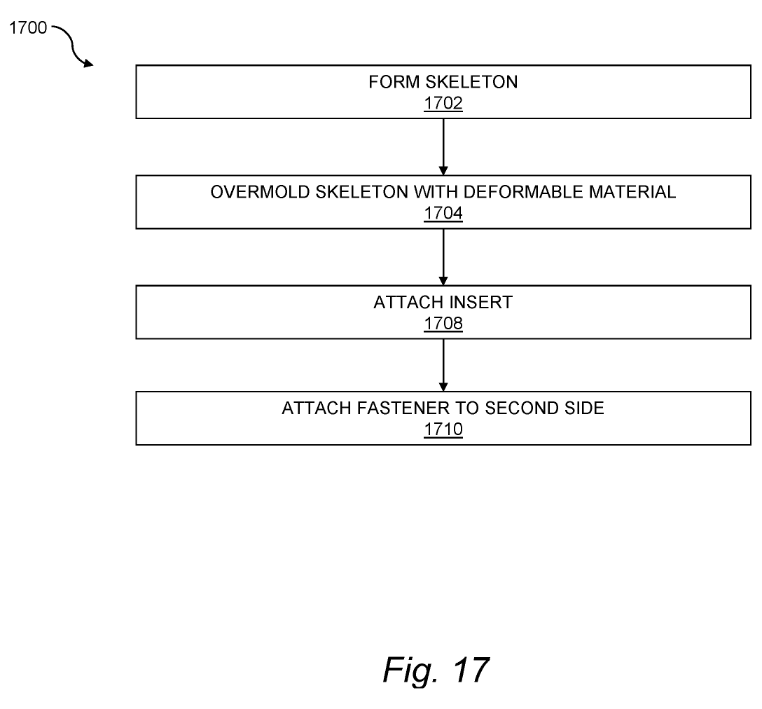

FIG. 17 is a flow chart of a method of manufacturing a container for physiological monitors. The method 1700 may be performed to manufacture any of the containers described herein.

As shown in a step 1702, the method 1700 may include forming a skeleton of a substantially rigid material. The substantially rigid material may include polycarbonate or similar materials as described herein. In certain aspects, forming the skeleton includes injection molding the substantially rigid material. For example, the skeleton may be formed as a first shot of material in an injection molding process. The skeleton may include a length that extends beyond the interior space in at least one dimension, as described herein. This configuration may provide a rigid backbone structure that is wider than the physiological monitor or device the container is configured to hold, which increases the normal force applied by garment elastic due to the increased surface area in communication with the garment when the container is coupled thereto.

As shown in a step 1704, the method 1700 may include overmolding at least a portion of the skeleton with a substantially deformable material to provide a deformable shell. The substantially deformable material may include silicone or similar materials as described herein. The deformable shell may have sidewalls extending from the skeleton that at least partially define an interior space shaped and sized to retain a physiological monitor. The overmolding process may be performed as a second shot of material in an injection molding process, where the deformable material adheres to the rigid material of the skeleton. In certain aspects, the method 1700 further includes forming one or more extensions that extend over at least a portion of the opening. The extensions may be formed as part of the deformable shell during the overmolding process, and may be configured to elastically deform to allow insertion and removal of a physiological monitor as described herein. In certain aspects, the method 1700 further includes forming a tab extending from a sidewall of the deformable shell. The tab may provide an alignment key for enforcing a predetermined position and orientation of the container in a guide, as described herein.

Overmolding the skeleton may include forming an opening in the overmolded material on a first side of the container for insertion and removal of the physiological monitor. The opening may be defined at least in part by the sidewalls of the deformable shell, and may be shaped and sized to allow a physiological monitor to pass therethrough and be retained within the interior space. The container design, including the rigid skeleton and the deformable shell, may make the overall assembly thinner compared to alternative configurations, improving comfort and wearability for a user.

As shown in a step 1708, the method 1700 may include attaching an insert to the skeleton. The insert may be configured to snap-fit, adhere, or mechanically lock into the skeleton, as described herein. The insert may provide a rigid backing plate and may include additional components such as a fastener, a communications component, and/or a charging component.

As shown in a step 1710, the method 1700 may include attaching a fastener to a second side of the container opposing the first side. The fastener may include a hook and loop fastener configured to engage with a complementary fastener on a wearable article. In certain aspects, the insert includes the fastener on an exterior surface thereof. In this configuration, attaching the insert to the skeleton as described in the step 1708 may also accomplish attaching the fastener to the second side of the container.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A system comprising:
a physiological monitor including one or more sensors on a sensing surface thereof and an RFID reader;
a garment with an RFID tag, a first hook and loop fastener over the RFID tag, and an alignment guide around the first hook and loop fastener; and
a container for removably and replaceably attaching the physiological monitor, the container including a rigid skeleton and a deformable shell overmolded onto the rigid skeleton, wherein the deformable shell includes:
an interior space shaped and sized to receive the physiological monitor,
an opening to the interior space on a first side of the container, the opening positioned to expose the one or more sensors through the opening when the physiological monitor is placed for use in the interior space, and
a second hook and loop fastener on a second side of the container, the second side of the container shaped and sized to fit within the alignment guide of the garment in a predetermined position and orientation when the first hook and loop fastener is coupled to the second hook and loop fastener,
wherein the predetermined position and orientation between the alignment guide and the second side of the container orients the RFID reader of the physiological monitor to retrieve data from the RFID tag of the garment.

2. The system of claim 1, wherein the one or more sensors of the physiological monitor include at least one of: an optical sensor, an electrical sensor, and a temperature sensor.

3. The system of claim 1, wherein the alignment guide includes a raised border forming an opening providing a mechanical key to align the container.

4. The system of claim 1, wherein the RFID tag encodes identifying information for the garment including at least one of: a location of an attachment region on the garment, a location of the attachment region relative to a body of a wearer when placed for use, a type of the garment, a size of the garment, and one or more preferred sensing operations for the garment or attachment region location.

5. The system of claim 1, wherein the container further comprises a tab extending from a sidewall of the deformable shell, the tab providing an alignment key for enforcing the predetermined position and orientation of the container within the alignment guide.

6. The system of claim 1, wherein the rigid skeleton extends beyond the interior space of the deformable shell in at least one dimension, thereby providing a rigid structure to the container extending beyond the physiological monitor in the at least one dimension when the physiological monitor is placed for use in the interior space.

7. The system of claim 1, wherein the rigid skeleton is formed of a polycarbonate, and wherein the deformable shell is formed of a silicone.

8. The system of claim 1, wherein the garment includes at least one of a shirt, pants, a compression sleeve, and an undergarment.

9. The system of claim 1, wherein the container includes one or more extensions that extend from a body of the container over at least a portion of the opening, wherein the one or more extensions are deformable to allow the physiological monitor to pass through the opening and to conform around the physiological monitor when disposed within the container.

10. The system of claim 1, wherein the physiological monitor protrudes a predetermined distance beyond the first side of the container when disposed within the container.

11. The system of claim 1, wherein the deformable shell has a predetermined tackiness that resists lateral movement of the container relative to skin of a wearer.

12. The system of claim 1, wherein the garment comprises a substantially elastic material structurally configured to apply a normal force directing the container and the physiological monitor into skin of a wearer, wherein the normal force results from hoop stress created by the garment urging the container toward the skin.

13. The system of claim 1, wherein the container comprises an insert configured to snap-fit, adhere, or mechanically lock into the rigid skeleton, wherein the insert includes the second hook and loop fastener on an exterior surface thereof.

14. The system of claim 1, further comprising a charging component configured to wirelessly charge the physiological monitor when the physiological monitor is placed for use in the interior space.

15. A method, comprising:
inserting a physiological monitor having one or more sensors on a sensing surface thereof into a container, the container including:
a first side and a second side, the first side defining an opening to an interior space shaped and sized to receive the physiological monitor, the second side including a fastener;
a rigid skeleton disposed at least along the second side of the container to form a backbone structure thereof; and
a deformable shell overmolded onto the skeleton and defining at least a portion of a perimeter of the opening to the interior space,
wherein the sensing surface is exposed through the opening when the physiological monitor is disposed within the container;
coupling the second side of the container to an attachment region of a wearable article, the attachment region including a complementary fastener to engage with the fastener of the container, the attachment region further including an alignment guide to provide a predetermined position and orientation of the container when coupled to the attachment region;
wearing the wearable article such that the sensing surface is disposed adjacent to skin of a wearer; and
performing a sensing operation using the one or more sensors of the physiological monitor.

16. The method of claim 15, wherein coupling the second side of the container to the attachment region of the wearable article includes aligning the container with the alignment guide, wherein the alignment guide includes a raised border having one or more features providing a mechanical key, and wherein aligning the container includes engaging a corresponding feature of the container with the mechanical key.

17. The method of claim 15, wherein the attachment region of the wearable article includes a data tag, and wherein the method further comprises reading data from the data tag using a data reader of the physiological monitor.

18. The method of claim 17, further comprising adapting operation of the physiological monitor based on the data.

19. The method of claim 15, further comprising generating a normal force pressing the container toward the skin of the wearer via hoop stress created by the wearable article.

20. The method of claim 15, further comprising removing the physiological monitor from the container, and replacing the physiological monitor to the container.
